SEQUENCE LISTING (15 sequences)

SEQ ID NO: 1 - DNA, 635 bp, Artificial Sequence
  Description of Artificial Sequence Synthetic polynucleotide
tgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 2 - DNA, 1205 bp, Artificial Sequence
  Description of Artificial Sequence Synthetic polynucleotide
ctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 3 - DNA, 1659 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttcagactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttctgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 4 - DNA, 512 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttc

SEQ ID NO: 5 - DNA, 1147 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttctgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 6 - DNA, 1843 bp, Artificial Sequence
  Description of Artificial Sequence Synthetic polynucleotide
gagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 7 - DNA, 1846 bp, Artificial Sequence
  Description of Artificial Sequence Synthetic polynucleotide
ctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccgtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgggatcc

SEQ ID NO: 8 - DNA, 386 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggctt

SEQ ID NO: 9 - DNA, 2355 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttcgagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 10 - DNA, 1021 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggctttgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 11 - DNA, 2229 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggcttgagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 12 - protein, 169 aa, Homo sapiens
MAPPWVPAMGFTLAPSLGCFVGSRFVHGEGLRWYAGLQKPSWHPPHWVLGPVWGTLYSAMGYGSYLVWKELGGFTEKAVVPLGLYTGQLALNWAWPPIFFGARQMGWALVDLLLVSGAAAATTVAWYQVSPLAARLLYPYLAWLAFATTLNYCVWRDNHGWHGGRRLPE

SEQ ID NO: 13 - DNA, 871 bp, Homo sapiens
ctgccaggcagtgcccttcccggagcgtgccctcgccgctgagctcccctgaacagcagctgcagcagccatggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtacggctcctacctggtctggaaagagctgggaggcttcacagagaaggctgtggttcccctgggcctctacactgggcagctggccctgaactgggcatggccccccatcttctttggtgcccgacaaatgggctgggccttggtggatctcctgctggtcagtggggcggcggcagccactaccgtggcctggtaccaggtgagcccgctggccgcccgcctgctctacccctacctggcctggctggccttcgcgaccacactcaactactgcgtatggcgggacaaccatggctggcatgggggacggcggctgccagagtgagtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggttgccccacctgagcccccacccgggagcagtgtcctgtgctttctgcatgcttagagcatgttcttggaacatggaattttataagctgaataaagtttttgacttcctttaaaaaaaaaaaaaaaaaaaaaaaaaaaaaaaaaaaaaaaaa

SEQ ID NO: 14 - DNA, 1591 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggcttctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 15 - DNA, 7827 bp, Artificial Sequence
  Description of Artificial Sequence Synthetic polynucleotide
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

(19) United States
(12) Patent Application Publication
(10) Pub. No.: US 2022/0348962 A1
(43) Pub. Date: Nov. 3, 2022

(54) MICROGLIA SPECIFIC PROMOTERS AND METHODS OF USE THEREFORE

(71) Applicants: Children's Medical Center Corporation, Boston, MA (US); Dana-Farber Cancer Institute, Inc., Boston, MA (US)

(72) Inventors: Alessandra BIFFI, Boston, MA (US); Valentina POLETTI, Boston, MA (US); Marco PEVIANI, Boston, MA (US); Francisco J. MOLINA-ESTEVEZ, Boston, MA (US)

(73) Assignees: Children's Medical Center Corporation, Boston, MA (US); Dana-Farber Cancer Institute, Inc., Boston, MA (US)

(21) Appl. No.: 17/765,796

(22) PCT Filed: Oct. 1, 2020
(86) PCT No.: PCT/US2020/053852

§ 371 Date: Mar. 31, 2022

Related U.S. Application Data
(60) Provisional application No. 62/908,966, filed on Oct. 1, 2019.

(51) Int. Cl. C12N 15/86

(52) U.S. Cl. CPC C12N 15/86; C12N 2740/15043; C12N 2830/008; A61K 48/00

(57) ABSTRACT

The present disclosure features methods and compositions for expressing transgenes in microglia. The disclosed compositions comprise isolated fragments of human and murine translocator protein (TSPO) promoters and expression cassettes comprising the same. The methods involve using these promoters and/or expression cassettes to express transgenes in a cell.

### CROSS-REFERENCE TO RELATED APPLICATIONS

This application claims the benefit of the following U.S. Provisional Application No.: 62/908,966, filed on Oct. 1, 2019, the entire contents of which are incorporated herein by reference.

### BACKGROUND OF THE INVENTION

Expression of transgenes in cell cultures has provided insights into cellular function and development. In vivo, expression transgenes encoding therapeutic polypeptides or a peptide to compensate for a naturally occurring deficiency is a promising approach for treating a subject having a condition that is amenable to traditional therapies. Vectors, such as lentiviral vectors, adenoviral vectors, and adeno-associated virus vectors, are used to deliver nucleic acid molecules encoding a transgene to its intended target (i.e., a cell) as well as regulatory sequences that facilitate expression of the transgene in the cell. These vectors have limited capacity, which limits the size of the transgenes that can be efficiently delivered to a cell. Accordingly, there is a pressing need for improved expression vectors and regulatory sequences for the delivery and expression of transgenes.

### SUMMARY OF THE INVENTION

As described below, the present invention features nucleic acid molecules containing truncated translocator protein (TSPO) promoters for expression of a transgene in a microglia cell.

In one aspect, the invention provides an isolated translocator protein (TSPO) promoter fragment sufficient to direct expression of a downstream polynucleotide in a microglial cell.

In one aspect, the invention provides an expression vector containing the promoter fragment of the above embodiment.

In one aspect, the invention provides an expression vector containing a regulatory sequence driving expression of a downstream gene, the regulatory sequence containing P1.

In one aspect, the invention provides an expression vector containing a regulatory sequence driving expression of a downstream gene, the regulatory sequence containing from 5′ to 3′ a P1 and P2.

In one aspect, the invention provides an expression vector containing a regulatory sequence driving expression of a downstream gene, the regulatory sequence containing from 5′ to 3′ P2 and P1.

In one aspect, the invention provides an expression vector containing a regulatory sequence driving expression of a downstream gene, the regulatory sequence containing from 5′ to 3′ El and P1.

In one aspect, the invention provides an expression vector containing a regulatory sequence driving expression of a downstream gene, the regulatory sequence containing from 5′ to 3′ E2 and P1.

In one aspect, the invention provides an expression vector containing a regulatory element containing a polynucleotide sequence having at least 85% identity to any of the following sequences:

>MPP01(hTSPO_prximal5′prom)(“P1”)
Tgcatcaccgcgttgcggcctcatcagtcccacgactttgtgcccattttactcatgaggagatggaggcccagagagccagtcaga
aagtggctgggccaggactaagagtgcagcgcgctgcctccgtgccctgcgtcaacagctcaaggaactggggtgctccggaaat
ggggccaaggctgctgggcagcaggacgctcagggccttggcctcaggagagcaaattccccactcggagatcggtcttgttgct
gcattttattcatgggaaatctgaggctagaagagacgacaaacgacacgccgttggacacacggcaacgttttagatgttgggtctg
gccgggcggccgtcaccggtcaccatggggaggaggaggagccgagagacttgctcgcggccggggggaggcagaagcgcg
tcccgcgggagaggtggctttgaggagtgagctcccggtcccgcggggacgcgagtgggcccagtgcccgggctgccaggcgg
ggcggggcggggccgggcgactgagaggggcggggcctggcggctgggaggggCGGGGCGGATGCGGGGA
CAGCGGCCTGGCTAACTCCTGCACGGCAGTGCCCTTCCCGGAGCGTGCCCTCGC
CG.
>MPP02(hTSPO_intronic5′prom)(“P2”)
CTGcacagtgaggacgggacgcggagggggcagcgggaacacgccgcccgcatggctgcgacagttggcagcgccgcgg
gacagagggaaactgaggccggagccgcagactggacacccgagggggcgacccggggcagcacttggggctcggctacgc
gcacagggggcggcgggcagcagagtctgggcctccgcggccggggttccaccgccggccgcctccggctcgcgcaacggg
agggaaaacttggacaaccctgccacgcccagcccttggccgcgtggcttctcctgctcgaagcgcggtcccaggagtggccgac
gctccctctcctgcccattccgcggatgggcaatcccaggcggaactcccttgagggtctcagaatatctgggagacctcgggctctt
gatctccgagacaccccgtttcgtagtggagaacagtccagatcggggaagtttattttgcccaaagccgcatagaggccccctggc
cctcgattccctctgcggggctcagcagcgttgcagcctagacgggtcttactgtgagccgagcagcctctgggaccacagaccttc
ccctaccccaacgttagaagccggagcccagcaaggagaagcgcgcacctcctgctgtgaacgcgcacgacgccagggcagct
gccagaggccatggcctggcgtgggcctggagcccctctggccagcctgcacggggccagggctacgggataccagcagcgtg
ccctgggctggatggcaggagagacaggacttgaggctgtcccagaatgggctcaggcagggcgaggatatcaggggaggtgg
tgtacaggaagcagccgcccagcttgcctggcacacagcaagccctgcccatgaaggcctactgccagaacagtgggcgaggcc
cggcgtctctgtggagtcggtggggcccgggacagggcagcctgaggcaggtttccactggcggtgaaaggggccgtgtggcaa
ggacaggagagccagcctcagcccagcaggggaaggcggcccctgagtctccacctggctgctggcagccccactcggagcat
cggcgaaactgaggcttgccaaagaagcctttgtccagagtcacgcagctggcgcggtggagccagggccagaacccgtgcagg
ctgatcccagcctgccttctccactgtgccccg
>E1
agactctgtctcaaaaaaaaaaaaaaaaaaaaaaaaagagagagagaaaaaggaagttagaaaaacagccctagaggccctacat
tctgagtaataggagttccagaaaggaagtgattgctgcacaacataaatttgaaaagaaagagaagtgagaaaatagagggaagg
aaatcaaagaaataatccaacttctgaaaagtaaagaatgagcttccagcgggaaagtgcctgttgagtgcaggcacagtggagga
aatgaagctgggtgtgttgccaggagttggaaacttgtctgagcaacatagcgcgaccctgtgtctacaaaaaaataaaaacaaaac
aaaaaacaaccaaagacttccgaaacagaatggctttagcctgctcaaccgcacactggcacctggccaacagcatctcttcatgatt
ctgaaggacaacgatctgcagctcagccaagcatcagccatctatggcctaggatgcaagaattcagcaatgttaccttcAgactct
gtctcaaaaaaaaaaaaaaaaaaaaaaaaagagagagagaaaaaggaagttagaaaaacagccctagaggccctacattctgagt
aataggagttccagaaaggaagtgattgctgcacaacataaatttgaaaagaaagagaagtgagaaaatagagggaaggaaatcaa
agaaataatccaacttctgaaaagtaaagaatgagcttccagcgggaaagtgcctgttgagtgcaggcacagtggaggaaatgaag
ctgggtgtgttgccaggagttggaaacttgtctgagcaacatagcgcgaccctgtgtctacaaaaaaataaaaacaaaacaaaaaac
aaccaaagacttccgaaacagaatggctttagcctgctcaaccgcacactggcacctggccaacagcatctcttcatgattctgaagg
acaacgatctgcagctcagccaagcatcagccatctatggcctaggatgcaagaattcagcaatgttaccttctgcatcaccgcgttg
cggcctcatcagtcccacgactttgtgcccattttactcatgaggagatggaggcccagagagccagtcagaaagtggctgggcca
ggactaagagtgcagcgcgctgcctccgtgccctgcgtcaacagctcaaggaactggggtgctccggaaatggggccaaggctg
ctgggcagcaggacgctcagggccttggcctcaggagagcaaattccccactcggagatcggtcttgttgctgcattttattcatggg
aaatctgaggctagaagagacgacaaacgacacgccgttggacacacggcaacgttttagatgttgggtctggccgggcggccgt
caccggtcaccatggggaggaggaggagccgagagacttgctcgcggccggggggaggcagaagcgcgtcccgcgggagag
gtggctttgaggagtgagctcccggtcccgcggggacgcgagtgggcccagtgcccgggctgccaggcggggcggggcgggg
ccgggcgactgagaggggcggggcctggcggctgggaggggCGGGGCGGATGCGGGGACAGCGGCC
TGGCTAACTCCTGCACGGCAGTGCCCTTCCCGGAGCGTGCCCTCGCCG
>E2
Tgcatcaccgcgttgcggcctcatcagtcccacgactttgtgcccattttactcatgaggagatggaggcccagagagccagtcaga
aagtggctgggccaggactaagagtgcagcgcgctgcctccgtgccctgcgtcaacagctcaaggaactggggtgctccggaaat
ggggccaaggctgctgggcagcaggacgctcagggccttggcctcaggagagcaaattccccactcggagatcggtcttgttgct
gcattttattcatgggaaatctgaggctagaagagacgacaaacgacacgccgttggacacacggcaacgttttagatgttgggtctg
gccgggcggccgtcaccggtcaccatggggaggaggaggagccgagagacttgctcgcggccggggggaggcagaagcgcg
tcccgcgggagaggtggctttgaggagtgagctcccggtcccgcggggacgcgagtgggcccagtgcccgggctgccaggcgg
ggcggggcggggccgggcgactgagaggggcggggcctggcggctgggaggggCGGGGCGGATGCGGGGA
CAGCGGCCTGGCTAACTCCTGCACGGCAGTGCCCTTCCCGGAGCGTGCCCTCGC
CG.
>MPP05A(hTSPO_upstream_enhancer)(“E1.1”)
agactctgtctcaaaaaaaaaaaaaaaaaaaaaaaaagagagagagaaaaaggaagttagaaaaacagccctagaggccctacat
tctgagtaataggagttccagaaaggaagtgattgctgcacaacataaatttgaaaagaaagagaagtgagaaaatagagggaagg
aaatcaaagaaataatccaacttctgaaaagtaaagaatgagcttccagcgggaaagtgcctgttgagtgcaggcacagtggagga
aatgaagctgggtgtgttgccaggagttggaaacttgtctgagcaacatagcgcgaccctgtgtctacaaaaaaataaaaacaaaac
aaaaaacaaccaaagacttccgaaacagaatggctttagcctgctcaaccgcacactggcacctggccaacagcatctcttcatgatt
ctgaaggacaacgatctgcagctcagccaagcatcagccatctatggcctaggatgcaagaattcagcaatgttaccttc
>MPP05B(hTSPO_upstream_enhancer)(“E1.2”)
Agactctgtctcaaaaaaaaaaaaaaaaaaaaaaaaagagagagagaaaaaggaagttagaaaaacagccctagaggccctaca
ttctgagtaataggagttccagaaaggaagtgattgctgcacaacataaatttgaaaagaaagagaagtgagaaaatagagggaag
gaaatcaaagaaataatccaacttctgaaaagtaaagaatgagcttccagcgggaaagtgcctgttgagtgcaggcacagtggagg
aaatgaagctgggtgtgttgccaggagttggaaacttgtctgagcaacatagcgcgaccctgtgtctacaaaaaaataaaaacaaaa
caaaaaacaaccaaagacttccgaaacagaatggctttagcctgctcaaccgcacactggcacctggccaacagcatctcttcatga
ttctgaaggacaacgatctgcagctcagccaagcatcagccatctatggcctaggatgcaagaattcagcaatgttaccttctgcatca
ccgcgttgcggcctcatcagtcccacgactttgtgcccattttactcatgaggagatggaggcccagagagccagtcagaaagtggc
tgggccaggactaagagtgcagcgcgctgcctccgtgccctgcgtcaacagctcaaggaactggggtgctccggaaatggggcc
aaggctgctgggcagcaggacgctcagggccttggcctcaggagagcaaattccccactcggagatcggtcttgttgctgcattttat
tcatgggaaatctgaggctagaagagacgacaaacgacacgccgttggacacacggcaacgttttagatgttgggtctggccgggc
ggccgtcaccggtcaccatggggaggaggaggagccgagagacttgctcgcggccggggggaggcagaagcgcgtcccgcg
ggagaggtggctttgaggagtgagctcccggtcccgcggggacgcgagtgggcccagtgcccgggctgccaggcggggcggg
gcggggccgggcgactgagaggggcggggcctggcggctgggaggggCGGGGCGGATGCGGGGACAGC
GGCCTGGCTAACTCCTGCACGGCAGTGCCCTTCCCGGAGCGTGCCCTCGCCG

In one aspect, the invention provides a mammalian cell containing the expression vector of any of the above aspects. In one embodiment, the mammalian cell is a rodent or human cell. In some embodiments, the mammalian cell is a hematopoietic stem or progenitor cell or a microglial cell.

In one aspect, the invention provides a method for expressing a therapeutic polypeptide in a cell, the method involving contacting the cell with an expression vector containing the promoter fragment of any of the above aspects or with the expression vector of any one of the above aspects.

In one aspect, the invention provides a method of treating a subject having a neurological disease, the method involving contacting a cell of the subject with an expression vector containing the promoter fragment of any of the above aspects operably linked to a transgene or with the expression vector of any of the above aspects containing a transgene.

In one aspect, the invention provides a method of treating a subject having a neurological disease, the method containing administering to the subject a cell containing an expression vector having the promoter fragment of any one of any of the above aspects or the expression vector of any of the above aspects.

In one aspect, the invention provides a pharmaceutical composition containing the expression vector of any of the above aspects.

In one aspect, the invention provides a pharmaceutical composition containing a cell of any one of the above aspects.

In any of the above aspects, the TSPO promoter fragment is responsive to inflammation. In any of the above aspects, the fragment is selected from one or more of the following: 300, 1200, 1600 nucleotides of a TSPO promoter. In any of the above aspects, the fragment contains at least about 300 nucleotides. In any of the above aspects, the promoter fragment has at least about 85% nucleotide sequence identity to the sequence of MPP01, MPP02, MPP03, or MPP04. In any of the above aspects, the full-length promoter contains nucleotides 83,518,714 to 83,521,414 of murine chromosome 15 or a corresponding portion of a human chromosome. In any of the above aspects, the fragment has at least about 95% sequence identity to the sequence of MPP01, MPP02, MPP03, or MPP04. In any of the above aspects, the promoter fragment contains MPP01.

In any of the above aspects, the promoter fragment contains a polynucleotide selected from one or more of the following:

>MPP01(hTSPO_prximal5′prom)(“Pl”)
Tgcatcaccgcgttgcggcctcatcagtcccacgactttgtgcccattttactcatgaggagatggaggcccagagagccagtcaga
aagtggctgggccaggactaagagtgcagcgcgctgcctccgtgccctgcgtcaacagctcaaggaactggggtgctccggaaat
ggggccaaggctgctgggcagcaggacgctcagggccttggcctcaggagagcaaattccccactcggagatcggtcttgttgct
gcattttattcatgggaaatctgaggctagaagagacgacaaacgacacgccgttggacacacggcaacgttttagatgttgggtctg
gccgggcggccgtcaccggtcaccatggggaggaggaggagccgagagacttgctcgcggccggggggaggcagaagcgcg
tcccgcgggagaggtggctttgaggagtgagctcccggtcccgcggggacgcgagtgggcccagtgcccgggctgccaggcgg
ggcggggcggggccgggcgactgagaggggcggggcctggcggctgggaggggCGGGGCGGATGCGGGGA
CAGCGGCCTGGCTAACTCCTGCACGGCAGTGCCCTTCCCGGAGCGTGCCCTCGC
CG
>MPP02(hTSPO_intronic5′prom)(“P2”)
CTGcacagtgaggacgggacgcggagggggcagcgggaacacgccgcccgcatggctgcgacagttggcagcgccgcgg
gacagagggaaactgaggccggagccgcagactggacacccgagggggcgacccggggcagcacttggggctcggctacgc
gcacagggggcggcgggcagcagagtctgggcctccgcggccggggttccaccgccggccgcctccggctcgcgcaacggg
agggaaaacttggacaaccctgccacgcccagcccttggccgcgtggcttctcctgctcgaagcgcggtcccaggagtggccgac
gctccctctcctgcccattccgcggatgggcaatcccaggcggaactcccttgagggtctcagaatatctgggagacctcgggctctt
gatctccgagacaccccgtttcgtagtggagaacagtccagatcggggaagtttattttgcccaaagccgcatagaggccccctggc
cctcgattccctctgcggggctcagcagcgttgcagcctagacgggtcttactgtgagccgagcagcctctgggaccacagaccttc
ccctaccccaacgttagaagccggagcccagcaaggagaagcgcgcacctcctgctgtgaacgcgcacgacgccagggcagct
gccagaggccatggcctggcgtgggcctggagcccctctggccagcctgcacggggccagggctacgggataccagcagcgtg
ccctgggctggatggcaggagagacaggacttgaggctgtcccagaatgggctcaggcagggcgaggatatcaggggaggtgg
tgtacaggaagcagccgcccagcttgcctggcacacagcaagccctgcccatgaaggcctactgccagaacagtgggcgaggcc
cggcgtctctgtggagtcggtggggcccgggacagggcagcctgaggcaggtttccactggcggtgaaaggggccgtgtggcaa
ggacaggagagccagcctcagcccagcaggggaaggcggcccctgagtctccacctggctgctggcagccccactcggagcat
cggcgaaactgaggcttgccaaagaagcctttgtccagagtcacgcagctggcgcggtggagccagggccagaacccgtgcagg
ctgatcccagcctgccttctccactgtgccccg
>MPP03(hTSPO_upstream_plus_intronic_prom)(“P1 + P2”)
GAGtgcatcaccgcgttgcggcctcatcagtcccacgactttgtgcccattttactcatgaggagatggaggcccagagagccagt
cagaaagtggctgggccaggactaagagtgcagcgcgctgcctccgtgccctgcgtcaacagctcaaggaactggggtgctccg
gaaatggggccaaggctgctgggcagcaggacgctcagggccttggcctcaggagagcaaattccccactcggagatcggtcttg
ttgctgcattttattcatgggaaatctgaggctagaagagacgacaaacgacacgccgttggacacacggcaacgttttagatgttgg
gtctggccgggcggccgtcaccggtcaccatggggaggaggaggagccgagagacttgctcgcggccggggggaggcagaa
gcgcgtcccgcgggagaggtggctttgaggagtgagctcccggtcccgcggggacgcgagtgggcccagtgcccgggctgcca
ggcggggcggggcggggccgggcgactgagaggggcggggcctggcggctgggaggggCGGGGCGGATGCG
GGGACAGCGGCCTGGCTAACTCCTGCACGGCAGTGCCCTTCCCGGAGCGTGCCC
TCGCCGCTGcacagtgaggacgggacgcggagggggcagcgggaacacgccgcccgcatggctgcgacagttggcag
cgccgcgggacagagggaaactgaggccggagccgcagactggacacccgagggggcgacccggggcagcacttggggctc
ggctacgcgcacagggggcggcgggcagcagagtctgggcctccgcggccggggttccaccgccggccgcctccggctcgcg
caacgggagggaaaacttggacaaccctgccacgcccagcccttggccgcgtggcttctcctgctcgaagcgcggtcccaggagt
ggccgacgctccctctcctgcccattccgcggatgggcaatcccaggcggaactcccttgagggtctcagaatatctgggagacctc
gggctcttgatctccgagacaccccgtttcgtagtggagaacagtccagatcggggaagtttattttgcccaaagccgcatagaggcc
ccctggccctcgattccctctgcggggctcagcagcgttgcagcctagacgggtcttactgtgagccgagcagcctctgggaccac
agaccttcccctaccccaacgttagaagccggagcccagcaaggagaagcgcgcacctcctgctgtgaacgcgcacgacgccag
ggcagctgccagaggccatggcctggcgtgggcctggagcccctctggccagcctgcacggggccagggctacgggataccag
cagcgtgccctgggctggatggcaggagagacaggacttgaggctgtcccagaatgggctcaggcagggcgaggatatcaggg
gaggtggtgtacaggaagcagccgcccagcttgcctggcacacagcaagccctgcccatgaaggcctactgccagaacagtggg
cgaggcccggcgtctctgtggagtcggtggggcccgggacagggcagcctgaggcaggtttccactggcggtgaaaggggccg
tgtggcaaggacaggagagccagcctcagcccagcaggggaaggcggcccctgagtctccacctggctgctggcagccccact
cggagcatcggcgaaactgaggcttgccaaagaagcctttgtccagagtcacgcagctggcgcggtggagccagggccagaacc
cgtgcaggctgatcccagcctgccttctccactgtgccccg
>MPP04(hTSPO_intronic_plus_upstream_prom)(“P2 + P1”)
CTGcacagtgaggacgggacgcggagggggcagcgggaacacgccgcccgcatggctgcgacagttggcagcgccgcgg
gacagagggaaactgaggccggagccgcagactggacacccgagggggcgacccggggcagcacttggggctcggctacgc
gcacagggggcggcgggcagcagagtctgggcctccgcggccggggttccaccgccggccgcctccggctcgcgcaacggg
agggaaaacttggacaaccctgccacgcccagcccttggccgcgtggcttctcctgctcgaagcgcggtcccaggagtggccgac
gctccctctcctgcccattccgcggatgggcaatcccaggcggaactcccttgagggtctcagaatatctgggagacctcgggctctt
gatctccgagacaccccgtttcgtagtggagaacagtccagatcggggaagtttattttgcccaaagccgcatagaggccccctggc
cctcgattccctctgcggggctcagcagcgttgcagcctagacgggtcttactgtgagccgagcagcctctgggaccacagaccttc
ccctaccccaacgttagaagccggagcccagcaaggagaagcgcgcacctcctgctgtgaacgcgcacgacgccagggcagct
gccagaggccatggcctggcgtgggcctggagcccctctggccagcctgcacggggccagggctacgggataccagcagcgtg
ccctgggctggatggcaggagagacaggacttgaggctgtcccagaatgggctcaggcagggcgaggatatcaggggaggtgg
tgtacaggaagcagccgcccagcttgcctggcacacagcaagccctgcccatgaaggcctactgccagaacagtgggcgaggcc
cggcgtctctgtggagtcggtggggcccgggacagggcagcctgaggcaggtttccactggcggtgaaaggggccgtgtggcaa
ggacaggagagccagcctcagcccagcaggggaaggcggcccctgagtctccacctggctgctggcagccccactcggagcat
cggcgaaactgaggcttgccaaagaagcctttgtccagagtcacgcagctggcgcggtggagccagggccagaacccgtgcagg
ctgatcccagcctgccttctccactgtgccccgtgcatcaccgcgttgcggcctcatcagtcccacgactttgtgcccattttactcatg
aggagatggaggcccagagagccagtcagaaagtggctgggccaggactaagagtgcagcgcgctgcctccgtgccctgcgtc
aacagctcaaggaactggggtgctccggaaatggggccaaggctgctgggcagcaggacgctcagggccttggcctcaggaga
gcaaattccccactcggagatcggtcttgttgctgcattttattcatgggaaatctgaggctagaagagacgacaaacgacacgccgtt
ggacacacggcaacgttttagatgttgggtctggccgggcggccgtcaccggtcaccatggggaggaggaggagccgagagact
tgctcgcggccggggggaggcagaagcgcgtcccgcgggagaggtggctttgaggagtgagctcccggtcccgcggggacgc
gagtgggcccagtgcccgggctgccaggcggggcggggcggggccgggcgactgagaggggcggggcctggcggctggga
ggggCGGGGCGGATGCGGGGACAGCGGCCTGGCTAACTCCTGCACGGCAGTGCC
CTTCCCGGAGCGTGCCCTCGCCGGGATCC

In any of the above aspects, the promoter fragment contains P1. In any of the above aspects, the promoter fragment contains P1+P2.

In any of the above aspects, the vector is a viral vector. In any of the above aspects, the viral vector is a lentiviral vector, gamma-retroviral vector, adenoviral vector, adeno-associated viral vector, or avian viral vector.

In any of the above aspects, the promoter fragment is operably linked to a transgene. In some embodiments, the transgene is a therapeutic polypeptide.

In any of the above aspects, the vector further contains an enhancer. In various embodiments, enhancer contains E1, E1.1, E1.2, or E2.

In any of the above aspects, the contacting is in vitro or in vivo. In any of the above aspects, the neurological disease is Alzheimer's disease, amyotrophic lateral sclerosis, metachromatic leuokodystrophy, adrenoleukodystophy, a lysosomal storage disorder, or Parkinson's disease.

In any of the above embodiments, the vector further contains an enhancer. In some embodiments, the enhancer contains E2. In some embodiments, the enhancer contains E1.

In any of the above aspects, the cell is a hematopoietic stem cell or progenitor thereof or a microglial cell.

In any of the above aspects, the subject undergoes ablative conditioning prior to the method. In some embodiments, ablative conditioning involves administering to the subject an alkylating agent. In some embodiments, the alkylating agent is busulfan. In some embodiments, the conditioning involves administering a CSF-1R inhibitor. In some embodiments, the inhibitor is PLX3397, PLX5622, or liposomal clodronate.

In any of the above aspects, the cell is a cell in vitro or in vivo. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, and a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining,” “assessing,” “assaying,” “measuring,” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of an expression vector of the invention required to direct expression of a therapeutic polypeptide in a cell of a subject.

By “enhancer” is meant a polynucleotide that increases transcription of a gene of interest. In one embodiment, the enhancer comprises 50-1,500 nucleotides. Exemplary enhancers useful in the methods of the invention include, but are not limited to sequences that comprising or having substantial identity to the following:

>MPP05A(hTSPO_upstream_enhancer)(“E1.1”)
agactctgtctcaaaaaaaaaaaaaaaaaaaaaaaaagagagagagaaaaaggaagttagaaaaacagccctagaggccctacattctgagt
aataggagttccagaaaggaagtgattgctgcacaacataaatttgaaaagaaagagaagtgagaaaatagagggaaggaaatcaaagaaata
atccaacttctgaaaagtaaagaatgagcttccagcgggaaagtgcctgttgagtgcaggcacagtggaggaaatgaagctgggtgtgttgcca
ggagttggaaacttgtctgagcaacatagcgcgaccctgtgtctacaaaaaaataaaaacaaaacaaaaaacaaccaaagacttccgaaacag
aatggctttagcctgctcaaccgcacactggcacctggccaacagcatctcttcatgattctgaaggacaacgatctgcagctcagccaagcatc
agccatctatggcctaggatgcaagaattcagcaatgttaccttc
>MPP05B(hTSPO_upstream_enhancer)(“E1.2”)
Agactctgtctcaaaaaaaaaaaaaaaaaaaaaaaaagagagagagaaaaaggaagttagaaaaacagccctagaggccctacattctgagt
aataggagttccagaaaggaagtgattgctgcacaacataaatttgaaaagaaagagaagtgagaaaatagagggaaggaaatcaaagaaata
atccaacttctgaaaagtaaagaatgagcttccagcgggaaagtgcctgttgagtgcaggcacagtggaggaaatgaagctgggtgtgttgcca
ggagttggaaacttgtctgagcaacatagcgcgaccctgtgtctacaaaaaaataaaaacaaaacaaaaaacaaccaaagacttccgaaacag
aatggctttagcctgctcaaccgcacactggcacctggccaacagcatctcttcatgattctgaaggacaacgatctgcagctcagccaagcatc
agccatctatggcctaggatgcaagaattcagcaatgttaccttctgcatcaccgcgttgcggcctcatcagtcccacgactttgtgcccattttact
catgaggagatggaggcccagagagccagtcagaaagtggctgggccaggactaagagtgcagcgcgctgcctccgtgccctgcgtcaac
agctcaaggaactggggtgctccggaaatggggccaaggctgctgggcagcaggacgctcagggccttggcctcaggagagcaaattcccc
actcggagatcggtcttgttgctgcattttattcatgggaaatctgaggctagaagagacgacaaacgacacgccgttggacacacggcaacgtt
ttagatgttgggtctggccgggcggccgtcaccggtcaccatggggaggaggaggagccgagagacttgctcgcggccggggggaggcag
aagcgcgtcccgcgggagaggtggctttgaggagtgagctcccggtcccgcggggacgcgagtgggcccagtgcccgggctgccaggcg
gggcggggcggggccgggcgactgagaggggcggggcctggcggctgggaggggCGGGGCGGATGCGGGGACAG
CGGCCTGGCTAACTCCTGCACGGCAGTGCCCTTCCCGGAGCGTGCCCTCGCCG
>E1
agactctgtctcaaaaaaaaaaaaaaaaaaaaaaaaagagagagagaaaaaggaagttagaaaaacagccctagaggccctacattctgagt
aataggagttccagaaaggaagtgattgctgcacaacataaatttgaaaagaaagagaagtgagaaaatagagggaaggaaatcaaagaaata
atccaacttctgaaaagtaaagaatgagcttccagcgggaaagtgcctgttgagtgcaggcacagtggaggaaatgaagctgggtgtgttgcca
ggagttggaaacttgtctgagcaacatagcgcgaccctgtgtctacaaaaaaataaaaacaaaacaaaaaacaaccaaagacttccgaaacag
aatggctttagcctgctcaaccgcacactggcacctggccaacagcatctcttcatgattctgaaggacaacgatctgcagctcagccaagcatc
agccatctatggcctaggatgcaagaattcagcaatgttaccttcAgactctgtctcaaaaaaaaaaaaaaaaaaaaaaaaagagagagagaa
aaaggaagttagaaaaacagccctagaggccctacattctgagtaataggagttccagaaaggaagtgattgctgcacaacataaatttgaaaa
gaaagagaagtgagaaaatagagggaaggaaatcaaagaaataatccaacttctgaaaagtaaagaatgagcttccagcgggaaagtgcctg
ttgagtgcaggcacagtggaggaaatgaagctgggtgtgttgccaggagttggaaacttgtctgagcaacatagcgcgaccctgtgtctacaaa
aaaataaaaacaaaacaaaaaacaaccaaagacttccgaaacagaatggctttagcctgctcaaccgcacactggcacctggccaacagcatc
tcttcatgattctgaaggacaacgatctgcagctcagccaagcatcagccatctatggcctaggatgcaagaattcagcaatgttaccttctgcatc
accgcgttgcggcctcatcagtcccacgactttgtgcccattttactcatgaggagatggaggcccagagagccagtcagaaagtggctgggcc
aggactaagagtgcagcgcgctgcctccgtgccctgcgtcaacagctcaaggaactggggtgctccggaaatggggccaaggctgctgggc
agcaggacgctcagggccttggcctcaggagagcaaattccccactcggagatcggtcttgttgctgcattttattcatgggaaatctgaggcta
gaagagacgacaaacgacacgccgttggacacacggcaacgttttagatgttgggtctggccgggcggccgtcaccggtcaccatggggag
gaggaggagccgagagacttgctcgcggccggggggaggcagaagcgcgtcccgcgggagaggtggctttgaggagtgagctcccggtc
ccgcggggacgcgagtgggcccagtgcccgggctgccaggcggggcggggcggggccgggcgactgagaggggcggggcctggcgg
ctgggaggggCGGGGCGGATGCGGGGACAGCGGCCTGGCTAACTCCTGCACGGCAGTGCCC
TTCCCGGAGCGTGCCCTCGCCG
>MPP06(hTSPO_intronic_enhancer)(“E2”)
Taggtggcttcacccctctgcctgagcctgagtcctgtccctgccaagactccgcccagccgacgcccaccccagctttccctggactcatccc
tcagcagatatctggatcctgcctagcctggctcagcatgactcatcatgcagggtaccgcccctgcccacctgttccccaataccgcaattcag
gagctgggcagttccccagaggccctaggaaactccccgcccccgaccaggctttctccactcctcccatctgaccgcctgttttctacgcctca
cgaccctctgagccccttggcgcactccgacataaccacagccaggcctgagaagccgccagcctccgcagcgagtgtgagcacgggactc
agaactggctt

In various embodiments the enhancer is E1, which contains E1.1 and E1.2. In various embodiments the E1 enhancer comprises the sequences E1.1+E1.2 or E1.2+E1.1 in the indicated order from 5′ to 3′.

Sequences comprising other regulatory elements useful in the methods of the invention follow:

>MPP03(hTSPO_upstream_enhancer_plus_upstream_and_intronic_prom)
agactctgtctcaaaaaaaaaaaaaaaaaaaaaaaaagagagagagaaaaaggaagttagaaaaacagccctagaggccctacattctgagt
aataggagttccagaaaggaagtgattgctgcacaacataaatttgaaaagaaagagaagtgagaaaatagagggaaggaaatcaaagaaata
atccaacttctgaaaagtaaagaatgagcttccagcgggaaagtgcctgttgagtgcaggcacagtggaggaaatgaagctgggtgtgttgcca
ggagttggaaacttgtctgagcaacatagcgcgaccctgtgtctacaaaaaaataaaaacaaaacaaaaaacaaccaaagacttccgaaacag
aatggctttagcctgctcaaccgcacactggcacctggccaacagcatctcttcatgattctgaaggacaacgatctgcagctcagccaagcatc
agccatctatggcctaggatgcaagaattcagcaatgttaccttcGAGtgcatcaccgcgttgcggcctcatcagtcccacgactttgtgcccat
tttactcatgaggagatggaggcccagagagccagtcagaaagtggctgggccaggactaagagtgcagcgcgctgcctccgtgccctgcgt
caacagctcaaggaactggggtgctccggaaatggggccaaggctgctgggcagcaggacgctcagggccttggcctcaggagagcaaatt
ccccactcggagatcggtcttgttgctgcattttattcatgggaaatctgaggctagaagagacgacaaacgacacgccgttggacacacggca
acgttttagatgttgggtctggccgggcggccgtcaccggtcaccatggggaggaggaggagccgagagacttgctcgcggccgggggga
ggcagaagcgcgtcccgcgggagaggtggctttgaggagtgagctcccggtcccgcggggacgcgagtgggcccagtgcccgggctgcc
aggcggggcggggcggggccgggcgactgagaggggcggggcctggcggctgggaggggCGGGGCGGATGCGGGGA
CAGCGGCCTGGCTAACTCCTGCACGGCAGTGCCCTTCCCGGAGCGTGCCCTCGCCGCTG
cacagtgaggacgggacgcggagggggcagcgggaacacgccgcccgcatggctgcgacagttggcagcgccgcgggacagagggaa
actgaggccggagccgcagactggacacccgagggggcgacccggggcagcacttggggctcggctacgcgcacagggggcggcggg
cagcagagtctgggcctccgcggccggggttccaccgccggccgcctccggctcgcgcaacgggagggaaaacttggacaaccctgccac
gcccagcccttggccgcgtggcttctcctgctcgaagcgcggtcccaggagtggccgacgctccctctcctgcccattccgcggatgggcaat
cccaggcggaactcccttgagggtctcagaatatctgggagacctcgggctcttgatctccgagacaccccgtttcgtagtggagaacagtcca
gatcggggaagtttattttgcccaaagccgcatagaggccccctggccctcgattccctctgcggggctcagcagcgttgcagcctagacgggt
cttactgtgagccgagcagcctctgggaccacagaccttcccctaccccaacgttagaagccggagcccagcaaggagaagcgcgcacctcc
tgctgtgaacgcgcacgacgccagggcagctgccagaggccatggcctggcgtgggcctggagcccctctggccagcctgcacggggcca
gggctacgggataccagcagcgtgccctgggctggatggcaggagagacaggacttgaggctgtcccagaatgggctcaggcagggcgag
gatatcaggggaggtggtgtacaggaagcagccgcccagcttgcctggcacacagcaagccctgcccatgaaggcctactgccagaacagt
gggcgaggcccggcgtctctgtggagtcggtggggcccgggacagggcagcctgaggcaggtttccactggcggtgaaaggggccgtgtg
gcaaggacaggagagccagcctcagcccagcaggggaaggcggcccctgagtctccacctggctgctggcagccccactcggagcatcg
gcgaaactgaggcttgccaaagaagcctttgtccagagtcacgcagctggcgcggtggagccagggccagaacccgtgcaggctgatccca
gcctgccttctccactgtgccccg
>MPP06-01 (hTSPO_prximal5′prom_plus_proximal_upstream_promoter)
Taggtggcttcacccctctgcctgagcctgagtcctgtccctgccaagactccgcccagccgacgcccaccccagctttccctggactcatccc
tcagcagatatctggatcctgcctagcctggctcagcatgactcatcatgcagggtaccgcccctgcccacctgttccccaataccgcaattcag
gagctgggcagttccccagaggccctaggaaactccccgcccccgaccaggctttctccactcctcccatctgaccgcctgttttctacgcctca
cgaccctctgagccccttggcgcactccgacataaccacagccaggcctgagaagccgccagcctccgcagcgagtgtgagcacgggactc
agaactggctttgcatcaccgcgttgcggcctcatcagtcccacgactttgtgcccattttactcatgaggagatggaggcccagagagccagtc
agaaagtggctgggccaggactaagagtgcagcgcgctgcctccgtgccctgcgtcaacagctcaaggaactggggtgctccggaaatggg
gccaaggctgctgggcagcaggacgctcagggccttggcctcaggagagcaaattccccactcggagatcggtcttgttgctgcattttattcat
gggaaatctgaggctagaagagacgacaaacgacacgccgttggacacacggcaacgttttagatgttgggtctggccgggcggccgtcacc
ggtcaccatggggaggaggaggagccgagagacttgctcgcggccggggggaggcagaagcgcgtcccgcgggagaggtggctttgag
gagtgagctcccggtcccgcggggacgcgagtgggcccagtgcccgggctgccaggcggggcggggcggggccgggcgactgagagg
ggcggggcctggcggctgggaggggCGGGGCGGATGCGGGGACAGCGGCCTGGCTAACTCCTGCAC
GGCAGTGCCCTTCCCGGAGCGTGCCCTCGCCG
>MPP03(hTSPO_upstream_plus_intronic_prom)
taggtggcttcacccctctgcctgagcctgagtcctgtccctgccaagactccgcccagccgacgcccaccccagctttccctggactcatccct
cagcagatatctggatcctgcctagcctggctcagcatgactcatcatgcagggtaccgcccctgcccacctgttccccaataccgcaattcagg
agctgggcagttccccagaggccctaggaaactccccgcccccgaccaggctttctccactcctcccatctgaccgcctgttttctacgcctcac
gaccctctgagccccttggcgcactccgacataaccacagccaggcctgagaagccgccagcctccgcagcgagtgtgagcacgggactca
gaactggcttGAGtgcatcaccgcgttgcggcctcatcagtcccacgactttgtgcccattttactcatgaggagatggaggcccagagagcc
agtcagaaagtggctgggccaggactaagagtgcagcgcgctgcctccgtgccctgcgtcaacagctcaaggaactggggtgctccggaaat
ggggccaaggctgctgggcagcaggacgctcagggccttggcctcaggagagcaaattccccactcggagatcggtcttgttgctgcattttat
tcatgggaaatctgaggctagaagagacgacaaacgacacgccgttggacacacggcaacgttttagatgttgggtctggccgggcggccgt
caccggtcaccatggggaggaggaggagccgagagacttgctcgcggccggggggaggcagaagcgcgtcccgcgggagaggtggcttt
gaggagtgagctcccggtcccgcggggacgcgagtgggcccagtgcccgggctgccaggcggggcggggcggggccgggcgactgag
aggggcggggcctggcggctgggaggggCGGGGCGGATGCGGGGACAGCGGCCTGGCTAACTCCTGC
ACGGCAGTGCCCTTCCCGGAGCGTGCCCTCGCCGCTGcacagtgaggacgggacgcggagggggcagc
gggaacacgccgcccgcatggctgcgacagttggcagcgccgcgggacagagggaaactgaggccggagccgcagactggacacccga
gggggcgacccggggcagcacttggggctcggctacgcgcacagggggcggcgggcagcagagtctgggcctccgcggccggggttcc
accgccggccgcctccggctcgcgcaacgggagggaaaacttggacaaccctgccacgcccagcccttggccgcgtggcttctcctgctcg
aagcgcggtcccaggagtggccgacgctccctctcctgcccattccgcggatgggcaatcccaggcggaactcccttgagggtctcagaatat
ctgggagacctcgggctcttgatctccgagacaccccgtttcgtagtggagaacagtccagatcggggaagtttattttgcccaaagccgcatag
aggccccctggccctcgattccctctgcggggctcagcagcgttgcagcctagacgggtcttactgtgagccgagcagcctctgggaccacag
accttcccctaccccaacgttagaagccggagcccagcaaggagaagcgcgcacctcctgctgtgaacgcgcacgacgccagggcagctgc
cagaggccatggcctggcgtgggcctggagcccctctggccagcctgcacggggccagggctacgggataccagcagcgtgccctgggct
ggatggcaggagagacaggacttgaggctgtcccagaatgggctcaggcagggcgaggatatcaggggaggtggtgtacaggaagcagcc
gcccagcttgcctggcacacagcaagccctgcccatgaaggcctactgccagaacagtgggcgaggcccggcgtctctgtggagtcggtgg
ggcccgggacagggcagcctgaggcaggtttccactggcggtgaaaggggccgtgtggcaaggacaggagagccagcctcagcccagca
ggggaaggcggcccctgagtctccacctggctgctggcagccccactcggagcatcggcgaaactgaggcttgccaaagaagcctttgtcca
gagtcacgcagctggcgcggtggagccagggccagaacccgtgcaggctgatcccagcctgccttctccactgtgccccg

“Exogenous nucleic acid molecule,” as used herein, refers to a nucleic acid molecule that is not an endogenous nucleic acid molecule, i.e., it is a nucleic acid molecule that does not naturally occur in a cell.

By “expression cassette” is meant those vector elements needed for expression of a gene. In one embodiment, an expression cassette comprises a promoter, a polynucleotide encoding a polypeptide of interest, and a terminator.

By “fragment” is meant a portion of a protein or nucleic acid that is substantially identical to a reference protein or nucleic acid. In some embodiments the portion retains at least 50%, 75%, or 80%, 85%, 90%, 95%, or even 99% of the biological activity of the reference protein or nucleic acid described herein. In one embodiment, a fragment comprises or consists of 200, 300, 600, 900, 1200, 1300, 1400, 1500, 1600, 1700, 1800 nucleotides of a reference sequence.

By “gene locus” is meant a position within a genome where a particular gene sequence is disposed.

By “hematopoietic stem cell (HSC)” is meant a stem cell that gives rise to a variety of blood cells.

By “hematopoietic stem progenitor cell (HSPC)” is meant a cell that gives rise to a hematopoietic stem cell.

Cells (e.g., HSCs, HSPCs) that may be used in conjunction with the compositions and methods described herein include CD34+/CD90+ cells, CD34+CD38− cells and CD34+/CD164+ cells. These cells may contain a higher percentage of HSCs or HSPCs. These cells are described in WO2015/059674, WO2017/218948, Radtke et al. Sci. Transl. Med. 9:1-10, 2017, Radtke et al. Mol Ther Methods Clin Dev.18:679-691, 2020, and Pellin et al. Nat. Comm. 10: 2395, 2019, the disclosures of each of which are hereby incorporated by reference in their entirety“.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by high-performance liquid chromatography analysis.

By “microglia” is meant an immune cell of the central nervous system.

By “nanoparticle” is meant a composite structure of nanoscale dimensions. In particular, nanoparticles are typically particles of a size in the range of from about 1 to about 1000 nm, and are usually spherical although different morphologies are possible depending on the nanoparticle composition. The portion of the nanoparticle contacting an environment external to the nanoparticle is generally identified as the surface of the nanoparticle. In nanoparticles herein described, the size limitation can be restricted to two dimensions and so that nanoparticles herein described include composite structure having a diameter from about 1 to about 1000 nm, where the specific diameter depends on the nanoparticle composition and on the intended use of the nanoparticle according to the experimental design. For example, nanoparticles to be used in several therapeutic applications typically have a size of about 200 nm or below, and the ones used, in particular, for delivery associated with therapeutic agents typically have a diameter from about 1 to about 100 nm.

By “neurological disease” is meant a disease affecting the central nervous system. In one embodiment, a neurological disease is associated with microglial loss or inflammation.

The term “nucleotide molecule”, “polynucleotide”, or “nucleic acid sequence” are used interchangeably to refer to a molecule comprising RNA or DNA. In various embodiments, the nucleotide molecule or polynucleotide comprises modified nucleotides (e.g., locked nucleic acids (LNA)). In some embodiments, the nucleotide molecule or polynucleotide comprises RNA and DNA. The sugar backbone of the nucleotide molecule is non-limiting and may comprise ribose, deoxyribose, or various other suitable sugars. In some embodiments, the nucleic acid molecule comprises at least two nucleotides covalently linked together. In some embodiments, the nucleic acid molecule of the present invention is single-stranded. In some embodiments, the nucleic acid molecule is double stranded. In some embodiments, the nucleic acid molecule is triple-stranded. In some embodiments, the nucleic acid molecule comprises phosphodiester bonds. In some embodiments, the nucleic acid molecule comprises a single-stranded or double-stranded deoxyribonucleic acid (DNA) or a single-stranded or double-stranded ribonucleic acid (RNA). In some embodiments, the nucleic acid molecule comprises a nucleic acid analog. In some embodiments, the nucleic acid analog has a backbone, comprising a bond other than and/or in addition to a phosphodiester bond, such as, by non-limiting example, phosphoramide, phosphorothioate, phosphorodithioate or O-methylphophoroamidite linkage. In some embodiments, the nucleic acid analog is selected from a nucleic acid analog with a backbone selected from a positive backbone; a non-ionic backbone and a non-ribose backbone. In some embodiments, the nucleic acid molecule contains one or more carbocyclic sugars. In some embodiments, the nucleic acid molecule comprises modifications of its ribose-phosphate backbone. In some embodiments, these modifications are performed to facilitate the addition of additional moieties, such as labels. In some embodiments, these modifications are performed to increase the stability and half-life of such molecules in physiological environments. In some embodiments, the term “polynucleotide” captures sequences that include any of the known base analogues of DNA and RNA such as, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein “neurodegenerative disease” refers to any of a group of diseases characterized by the progressive loss of structure and/or function of neurons, including death of neurons. Exemplary neurodegenerative diseases include, without limitation, Alzheimer's disease, Parkinson's disease, metachromatic leuokodystrophy, adrenoleukodystophy, other lysosomal storage disorders, and amyotrophic lateral sclerosis.

As used herein, “obtaining” as in “obtaining an agent” includes synthesizing, purchasing, or otherwise acquiring the agent.

By “PLX3397” is meant a colony stimulating factor-1 receptor (CSF-1R) inhibitor having the structure

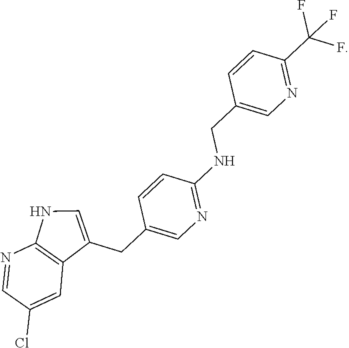 [SMILES: FC(F)(F)c1ccc(CNc2ccc(Cc3c[nH]c4ncc(Cl)cc34)cn2)cn1]

By “PLX5622” is meant a colony stimulating factor-1 receptor (CSF-1R) inhibitor having the structure

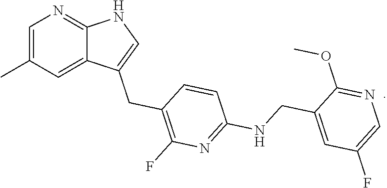 [SMILES: COc1ncc(F)cc1CNc1ccc(Cc2c[nH]c3ncc(C)cc23)c(F)n1]

By “operably linked” is meant that a first polynucleotide is positioned adjacent to a second polynucleotide that directs transcription of the first polynucleotide.

As used herein, the terms “prevent,” “preventing,” “prevention,” “prophylactic treatment,” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be, in some embodiments at least about 16 amino acids, at least about 20 amino acids, at least about 25 amino acids, or about 35 amino acids, about 50 amino acids, or about 100 amino acids, or any integer thereabout or therebetween. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In one embodiment, a reference sequence is the sequence of a full-length TSPO promoter.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or about 250 mM NaCl and 25 mM trisodium citrate. Low stringency hybridization can be obtained in the absence of organic solvent, e.g., formamide, while high stringency hybridization can be obtained in the presence of at least about 35% formamide, and in some embodiments, at least about 50% formamide. Stringent temperature conditions will ordinarily include temperatures of at least about 30° C. at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will comprise less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In some embodiments, wash steps will occur at 25° C. in 30 mM NaCl, 3 mM trisodium citrate, and 0.1% SDS. In other embodiments, wash steps will occur at 42 C in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS.

Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In the context of a TSPO promoter, “responsive to inflammation” means that promoter activity increases as inflammation increases.

By “subject” is meant a mammal, including, but not limited to, a human or non-human mammal, such as a murine, bovine, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transgene” is meant an exogenous nucleic acid molecule, introduced into a host cell, that encodes a polypeptide or polynucleotide to be expressed in the host cell.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “translocator protein (TSPO)” or “TSPO polypeptide” is meant a polypeptide or fragment thereof having at least about 85% amino acid sequence identity to GenBank Accession No. AAH01110.1 and having cholesterol transport activity. An exemplary TSPO polypeptide sequence is provided below.
1 mappwvpamg ftlapslgcf vgsrfvhgeg lrwyaglqkp swhpphwvlg pvwgtlysam
61 gygsylvwke lggftekavv plglytgqla lnwawppiff garqmgwalv dlllvsgaaa
121 attvawyqvs plaarllypy lawlafattl nycvwrdnhg whggrrlpe

By “translocator protein (TSPO) polynucleotide” is meant a nucleic acid molecule encoding a TSPO polypeptide. An exemplary TSPO polynucleotide sequence is provided below.

>BC001110.2 Homo sapiens translocator protein
(18 kDa), mRNA (cDNA clone MGC: 1184 IMAGE:
2989070), complete cds
CTGCCAGGCAGTGCCCTTCCCGGAGCGTGCCCTCGCCGCTGAGCTCCCCT
GAACAGCAGCTGCAGCAGCCATGGCCCCGCCCTGGGTGCCCGCCATGGGC
TTCACGCTGGCGCCCAGCCTGGGGTGCTTCGTGGGCTCCCGCTTTGTCCA
CGGCGAGGGTCTCCGCTGGTACGCCGGCCTGCAGAAGCCCTCGTGGCACC
CGCCCCACTGGGTGCTGGGCCCTGTCTGGGGCACGCTCTACTCAGCCATG
GGGTACGGCTCCTACCTGGTCTGGAAAGAGCTGGGAGGCTTCACAGAGAA
GGCTGTGGTTCCCCTGGGCCTCTACACTGGGCAGCTGGCCCTGAACTGGG
CATGGCCCCCCATCTTCTTTGGTGCCCGACAAATGGGCTGGGCCTTGGTG
GATCTCCTGCTGGTCAGTGGGGCGGCGGCAGCCACTACCGTGGCCTGGTA
CCAGGTGAGCCCGCTGGCCGCCCGCCTGCTCTACCCCTACCTGGCCTGGC
TGGCCTTCGCGACCACACTCAACTACTGCGTATGGCGGGACAACCATGGC
TGGCATGGGGGACGGCGGCTGCCAGAGTGAGTGCCCGGCCCACCAGGGAC
TGCAGCTGCACCAGCAGGTGCCATCACGCTTGTGATGTGGTGGCCGTCAC
GCTTTCATGACCACTGGGCCTGCTAGTCTGTCAGGGCCTTGGCCCAGGGG
TCAGCAGAGCTTCAGAGGTTGCCCCACCTGAGCCCCCACCCGGGAGCAGT
GTCCTGTGCTTTCTGCATGCTTAGAGCATGTTCTTGGAACATGGAATTTT
ATAAGCTGAATAAAGTTTTTGACTTCCTTTAAAAAAAAAAAAAAAAAAAA
AAAAAAAAAAAAAAAAAAAAA

By “translocator protein promoter” or “TSPO promoter” is meant a polynucleotide sufficient to direct expression of a transgene in a microglial cell. In one embodiment the TSPO promoter is responsive to inflammation. Exemplary promoters useful in the methods of the invention include, but are not limited to, the following:

>MPP01(hTSPO_prximal5′prom)(“P1”)
Tgcatcaccgcgttgcggcctcatcagtcccacgactttgtgcccatttt
actcatgaggagatggaggcccagagagccagtcagaaagtggctgggcc
aggactaagagtgcagcgcgctgcctccgtgccctgcgtcaacagctcaa
ggaactggggtgctccggaaatggggccaaggctgctgggcagcaggacg
ctcagggccttggcctcaggagagcaaattccccactcggagatcggtct
tgttgctgcattttattcatgggaaatctgaggctagaagagacgacaaa
cgacacgccgttggacacacggcaacgttttagatgttgggtctggccgg
gcggccgtcaccggtcaccatggggaggaggaggagccgagagacttgct
cgcggccggggggaggcagaagcgcgtcccgcgggagaggtggctttgag
gagtgagctcccggtcccgcggggacgcgagtgggcccagtgcccgggct
gccaggcggggcggggcggggccgggcgactgagaggggcggggcctggc
ggctgggaggggCGGGGCGGATGCGGGGACAGCGGCCTGGCTAACTCCTG
CACGGCAGTGCCCTTCCCGGAGCGTGCCCTCGCCG

The P1 promoter comprises 635 bp that correspond to nucleotide residues −562 to +73 (capital letters) of the hTspo immediate 5′ promoter of the hTspo immediate 5′ promoter.

>MPP02(hTSPO_intronic5′prom)(“P2”)
CTGcacagtgaggacgggacgcggagggggcagcgggaacacgccgcccgcatggctgcgacagttggcagcgccgcgggacagag
ggaaactgaggccggagccgcagactggacacccgagggggcgacccggggcagcacttggggctcggctacgcgcacagggggcgg
cgggcagcagagtctgggcctccgcggccggggttccaccgccggccgcctccggctcgcgcaacgggagggaaaacttggacaaccctg
ccacgcccagcccttggccgcgtggcttctcctgctcgaagcgcggtcccaggagtggccgacgctccctctcctgcccattccgcggatggg
caatcccaggcggaactcccttgagggtctcagaatatctgggagacctcgggctcttgatctccgagacaccccgtttcgtagtggagaacagt
ccagatcggggaagtttattttgcccaaagccgcatagaggccccctggccctcgattccctctgcggggctcagcagcgttgcagcctagacg
ggtcttactgtgagccgagcagcctctgggaccacagaccttcccctaccccaacgttagaagccggagcccagcaaggagaagcgcgcac
ctcctgctgtgaacgcgcacgacgccagggcagctgccagaggccatggcctggcgtgggcctggagcccctctggccagcctgcacggg
gccagggctacgggataccagcagcgtgccctgggctggatggcaggagagacaggacttgaggctgtcccagaatgggctcaggcaggg
cgaggatatcaggggaggtggtgtacaggaagcagccgcccagcttgcctggcacacagcaagccctgcccatgaaggcctactgccagaa
cagtgggcgaggcccggcgtctctgtggagtcggtggggcccgggacagggcagcctgaggcaggtttccactggcggtgaaaggggccg
tgtggcaaggacaggagagccagcctcagcccagcaggggaaggcggcccctgagtctccacctggctgctggcagccccactcggagca
tcggcgaaactgaggcttgccaaagaagcctttgtccagagtcacgcagctggcgcggtggagccagggccagaacccgtgcaggctgatc
ccagcctgccttctccactgtgccccg
>MPP03(hTSPO_upstream_plus_intronic_prom)(“P1 + P2”)
GAGtgcatcaccgcgttgcggcctcatcagtcccacgactttgtgcccattttactcatgaggagatggaggcccagagagccagtcagaaa
gtggctgggccaggactaagagtgcagcgcgctgcctccgtgccctgcgtcaacagctcaaggaactggggtgctccggaaatggggccaa
ggctgctgggcagcaggacgctcagggccttggcctcaggagagcaaattccccactcggagatcggtcttgttgctgcattttattcatgggaa
atctgaggctagaagagacgacaaacgacacgccgttggacacacggcaacgttttagatgttgggtctggccgggcggccgtcaccggtca
ccatggggaggaggaggagccgagagacttgctcgcggccggggggaggcagaagcgcgtcccgcgggagaggtggctttgaggagtg
agctcccggtcccgcggggacgcgagtgggcccagtgcccgggctgccaggcggggcggggcggggccgggcgactgagaggggcg
gggcctggcggctgggaggggCGGGGCGGATGCGGGGACAGCGGCCTGGCTAACTCCTGCACGG
CAGTGCCCTTCCCGGAGCGTGCCCTCGCCGCTGcacagtgaggacgggacgcggagggggcagcgggaaca
cgccgcccgcatggctgcgacagttggcagcgccgcgggacagagggaaactgaggccggagccgcagactggacacccgagggggcg
acccggggcagcacttggggctcggctacgcgcacagggggcggcgggcagcagagtctgggcctccgcggccggggttccaccgccg
gccgcctccggctcgcgcaacgggagggaaaacttggacaaccctgccacgcccagcccttggccgcgtggcttctcctgctcgaagcgcg
gtcccaggagtggccgacgctccctctcctgcccattccgcggatgggcaatcccaggcggaactcccttgagggtctcagaatatctgggag
acctcgggctcttgatctccgagacaccccgtttcgtagtggagaacagtccagatcggggaagtttattttgcccaaagccgcatagaggcccc
ctggccctcgattccctctgcggggctcagcagcgttgcagcctagacgggtcttactgtgagccgagcagcctctgggaccacagaccttccc
ctaccccaacgttagaagccggagcccagcaaggagaagcgcgcacctcctgctgtgaacgcgcacgacgccagggcagctgccagagg
ccatggcctggcgtgggcctggagcccctctggccagcctgcacggggccagggctacgggataccagcagcgtgccctgggctggatgg
caggagagacaggacttgaggctgtcccagaatgggctcaggcagggcgaggatatcaggggaggtggtgtacaggaagcagccgccca
gcttgcctggcacacagcaagccctgcccatgaaggcctactgccagaacagtgggcgaggcccggcgtctctgtggagtcggtggggccc
gggacagggcagcctgaggcaggtttccactggcggtgaaaggggccgtgtggcaaggacaggagagccagcctcagcccagcagggga
aggcggcccctgagtctccacctggctgctggcagccccactcggagcatcggcgaaactgaggcttgccaaagaagcctttgtccagagtca
cgcagctggcgcggtggagccagggccagaacccgtgcaggctgatcccagcctgccttctccactgtgccccg
>MPP04(hTSPO_intronic_plus_upstream_prom)(“P2 + Pl”)
CTGcacagtgaggacgggacgcggagggggcagcgggaacacgccgcccgcatggctgcgacagttggcagcgccgcgggacagag
ggaaactgaggccggagccgcagactggacacccgagggggcgacccggggcagcacttggggctcggctacgcgcacagggggcgg
cgggcagcagagtctgggcctccgcggccggggttccaccgccggccgcctccggctcgcgcaacgggagggaaaacttggacaaccctg
ccacgcccagcccttggccgcgtggcttctcctgctcgaagcgcggtcccaggagtggccgacgctccctctcctgcccattccgcggatggg
caatcccaggcggaactcccttgagggtctcagaatatctgggagacctcgggctcttgatctccgagacaccccgtttcgtagtggagaacagt
ccagatcggggaagtttattttgcccaaagccgcatagaggccccctggccctcgattccctctgcggggctcagcagcgttgcagcctagacg
ggtcttactgtgagccgagcagcctctgggaccacagaccttcccctaccccaacgttagaagccggagcccagcaaggagaagcgcgcac
ctcctgctgtgaacgcgcacgacgccagggcagctgccagaggccatggcctggcgtgggcctggagcccctctggccagcctgcacggg
gccagggctacgggataccagcagcgtgccctgggctggatggcaggagagacaggacttgaggctgtcccagaatgggctcaggcaggg
cgaggatatcaggggaggtggtgtacaggaagcagccgcccagcttgcctggcacacagcaagccctgcccatgaaggcctactgccagaa
cagtgggcgaggcccggcgtctctgtggagtcggtggggcccgggacagggcagcctgaggcaggtttccactggcggtgaaaggggccg
tgtggcaaggacaggagagccagcctcagcccagcaggggaaggcggcccctgagtctccacctggctgctggcagccccactcggagca
tcggcgaaactgaggcttgccaaagaagcctttgtccagagtcacgcagctggcgcggtggagccagggccagaacccgtgcaggctgatc
ccagcctgccttctccactgtgccccgtgcatcaccgcgttgcggcctcatcagtcccacgactttgtgcccattttactcatgaggagatggagg
cccagagagccagtcagaaagtggctgggccaggactaagagtgcagcgcgctgcctccgtgccctgcgtcaacagctcaaggaactgggg
tgctccggaaatggggccaaggctgctgggcagcaggacgctcagggccttggcctcaggagagcaaattccccactcggagatcggtcttg
ttgctgcattttattcatgggaaatctgaggctagaagagacgacaaacgacacgccgttggacacacggcaacgttttagatgttgggtctggcc
gggcggccgtcaccggtcaccatggggaggaggaggagccgagagacttgctcgcggccggggggaggcagaagcgcgtcccgcggg
agaggtggctttgaggagtgagctcccggtcccgcggggacgcgagtgggcccagtgcccgggctgccaggcggggcggggcggggcc
gggcgactgagaggggcggggcctggcggctgggaggggCGGGGCGGATGCGGGGACAGCGGCCTGGCTA
ACTCCTGCACGGCAGTGCCCTTCCCGGAGCGTGCCCTCGCCGGGATCC
>MPP02(hTSPO_intronic5′prom)
taggtggcttcacccctctgcctgagcctgagtcctgtccctgccaagactccgcccagccgacgcccaccccagctttccctggactcatccct
cagcagatatctggatcctgcctagcctggctcagcatgactcatcatgcagggtaccgcccctgcccacctgttccccaataccgcaattcagg
agctgggcagttccccagaggccctaggaaactccccgcccccgaccaggctttctccactcctcccatctgaccgcctgttttctacgcctcac
gaccctctgagccccttggcgcactccgacataaccacagccaggcctgagaagccgccagcctccgcagcgagtgtgagcacgggactca
gaactggcttCTGcacagtgaggacgggacgcggagggggcagcgggaacacgccgcccgcatggctgcgacagttggcagcgccgc
gggacagagggaaactgaggccggagccgcagactggacacccgagggggcgacccggggcagcacttggggctcggctacgcgcaca
gggggcggcgggcagcagagtctgggcctccgcggccggggttccaccgccggccgcctccggctcgcgcaacgggagggaaaacttg
gacaaccctgccacgcccagcccttggccgcgtggcttctcctgctcgaagcgcggtcccaggagtggccgacgctccctctcctgcccattc
cgcggatgggcaatcccaggcggaactcccttgagggtctcagaatatctgggagacctcgggctcttgatctccgagacaccccgtttcgtag
tggagaacagtccagatcggggaagtttattttgcccaaagccgcatagaggccccctggccctcgattccctctgcggggctcagcagcgttg
cagcctagacgggtcttactgtgagccgagcagcctctgggaccacagaccttcccctaccccaacgttagaagccggagcccagcaaggag
aagcgcgcacctcctgctgtgaacgcgcacgacgccagggcagctgccagaggccatggcctggcgtgggcctggagcccctctggccag
cctgcacggggccagggctacgggataccagcagcgtgccctgggctggatggcaggagagacaggacttgaggctgtcccagaatgggc
tcaggcagggcgaggatatcaggggaggtggtgtacaggaagcagccgcccagcttgcctggcacacagcaagccctgcccatgaaggcc
tactgccagaacagtgggcgaggcccggcgtctctgtggagtcggtggggcccgggacagggcagcctgaggcaggtttccactggcggtg
aaaggggccgtgtggcaaggacaggagagccagcctcagcccagcaggggaaggcggcccctgagtctccacctggctgctggcagccc
cactcggagcatcggcgaaactgaggcttgccaaagaagcctttgtccagagtcacgcagctggcgcggtggagccagggccagaacccgt
gcaggctgatcccagcctgccttctccactgtgccccg

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a diagram that identifies a non-exclusive list of neurodegenerative diseases that have been or could be susceptible to be cured or benefited by human stem cell gene therapy. The therapy includes obtaining human stem cells from a subject (1) and modifying these cells ex vivo to express a therapeutic transgene (2). The modified cells are then reinfused into a patient in need thereof (3), and the stem cells repopulate a central nervous system cell population (4).

FIGS. 2A and 2B show the full mTspo promoter structure and function. FIG. 2A is a schematic diagram of the structure of the lentiviral vector transfer plasmid T2700 containing 2.7 kb upstream from mTspo, placed upstream the GFP reporter. FIG. 2B is a graph depicting GFP expression driven by the 2.7 kb Tspo promoter sequence increased upon LPS stimulation of transduced BV-2 cells.

FIG. 3 is a diagram of the mTspo promoters and the expression cassette in the cloned vectors. “LTR” is an abbreviation for long terminal repeat; “GFP” is an abbreviation for green fluorescent protein; “wPRE” is an abbreviation for woodchuck hepatitis virus post-transcriptional regulatory element. These acronyms are used throughout the specification and the drawings.

FIG. 4 is a graph of the lentiviral vector titers observed for the T0300, T1200, T1600, and T2700 transfer plasmids comprising approximately 300 base pairs, 1200 base pairs, 1600 base pairs, and full-length TSPO promoters, respectively. Titers were higher for the smaller promoters.

FIGS. 5A and 5B show differential expression of the mTspo lentiviral vectors in different cell lines. FIG. 5A is a graph depicting a drastic drop in the mean fluorescent intensity (MFI) measured in arbitrary units (a.u.) in NIH-3T3 fibroblasts after deleting the distal section of the promoter. FIG. 5B is a graph showing the MFI observed for the different lentiviral vectors in BV-2 cells. The same distal deletion did not affect substantially the expression in microglia BV-2 cells, unlike that observed in FIG. 5A.

FIGS. 6A to 6C show GFP expression modulation upon LPS stimulation. FIG. 6A is a graph characterizing the mean fluorescence intensity fold change of GFP expression in NIH-3T3 cells stably transduced with the different mTspo reporter vectors. FIG. 6B is a graph depicting the mean fluorescence intensity fold change of GFP expression in BV-2 cells stably transduced with the different mTspo reporter vectors. FIG. 6C is a graph depicting the fold change of mTspo mRNA in BV-2 cells exposed to LPS for 24 hours.

FIG. 7 is a schematic diagram showing the experimental outline for the validation of mTspo promoter for in vivo delivery. “dpt” means days post transduction.

FIGS. 8A and 8B show HSPC transduction efficiency by the lentiviral vectors carrying mTSPO promoter variants. FIG. 8A is a graph of the percentage of GFP⁺ cells in liquid culture progeny of the HSPCs transduced with the indicated vector as detected by flow cytometry. FIG. 8B is a graph depicting the vector copy number (VCN) per cell present in the progeny of the HSPCs transduced with the indicated vector. “UT” refers to untransduced cells; PGK.GFP refers to cells transduced with a non-inducible PGK-GFP control.

FIGS. 9A-9C shows the analysis of GFP expression in brain myeloid cells from animals transplanted with the mTSPO LVs, 45 days post-transplant. FIG. 9A is a series of scatter plots of flow cytometry data that depict a gating strategy for brain cell analysis at flow. Cells positive for the donor CD45.1 marker within the population of cells positive for CD11b, in the fraction of viable cells that are negative for the vital staining, can be used to evaluate the strength of the promoters under testing. FIG. 9B is a graph showing GFP expression in donor-derived brain myeloid cells of un-stimulated versus LPS induced transplant recipients comprising a control PGK-GFP vector. FIG. 9C is a graph showing GFP expression in donor-derived brain myeloid cells of unstimulated versus LPS induced transplant recipients comprising the T1600-GFP promoter.

FIG. 10 is an image of a web-browser illustrating the portion of human chromosome 22 that contains the hTspo gene. Methylation patterns, the relationship between enhancers predicted and the density of transcription factor binding domains allowed identification of a short (approximately 500 bp) 5′ proximal promoter (P1), an extended intragenic promoter (P2) comprising about 1 kb, and several enhancers either 5′ (E1.1 and E1.2) or 3′ sequences (E2).

FIG. 11 is the schematic representation of the intended viral vector harboring the different promoters.

FIG. 12 is Chip-seq genomic maps relating to identification of putative translocator protein (TSPO) transcriptional regulatory elements.

FIG. 13 is vector maps for five lentiviral vectors carrying a translocator protein-based (TSPO-based) promoter/enhancer.

FIG. 14 is a bar graph relating to HMC3 transduced with the indicated lentiviral vectors (LVs) at a MOI (Multiplicity of Infection) of 5. The bars show the number of resulting integrated vectors (alternatively, vector copy number (VCN)) two weeks post-transduction. P.GFP vector represents the control lentiviral vector (LV) carrying the ubiquitous human phosphoglycerate kinase (PGK) promoter.

FIGS. 15A and 15B are a bar graph and a plot demonstrating that HMC3 cells overexpress MHC-II (major histocompatibility complex II) and translocator protein (TSPO) (N=2 experiments).

FIGS. 16A to 16C are plots and a bar graph relating to HMC3 transduced with the lentiviral vectors (LVs) and activated to overexpress MHC II (major histocompatibility complex II), translocator protein (TSPO), and green fluorescent protein (GFP). MFI means Mean Fluorescence Intensity as detected by Fluorescence Activated Cell Sorting (FACS).

FIG. 17 is a heat map for mRNA expression driven by the lentiviral vectors (LVs) upon cell activation. In the heatmap, mRNA amount is color-coded according to the scale provided to the right of the heat map of FIG. 17. The two columns corresponding to each gene represent the mRNA expression before (left) and after (right) cell-activation. Numeric values in the cells indicate the mRNA expression of the three analyzed genes (major histocompatibility complex II (MHC-II), translocator protein (TSPO) and green fluorescent protein (GFP)) normalized to the human GAPDH (glyceraldehyde 3-phosphate dehydrogenase).

### DETAILED DESCRIPTION OF THE INVENTION

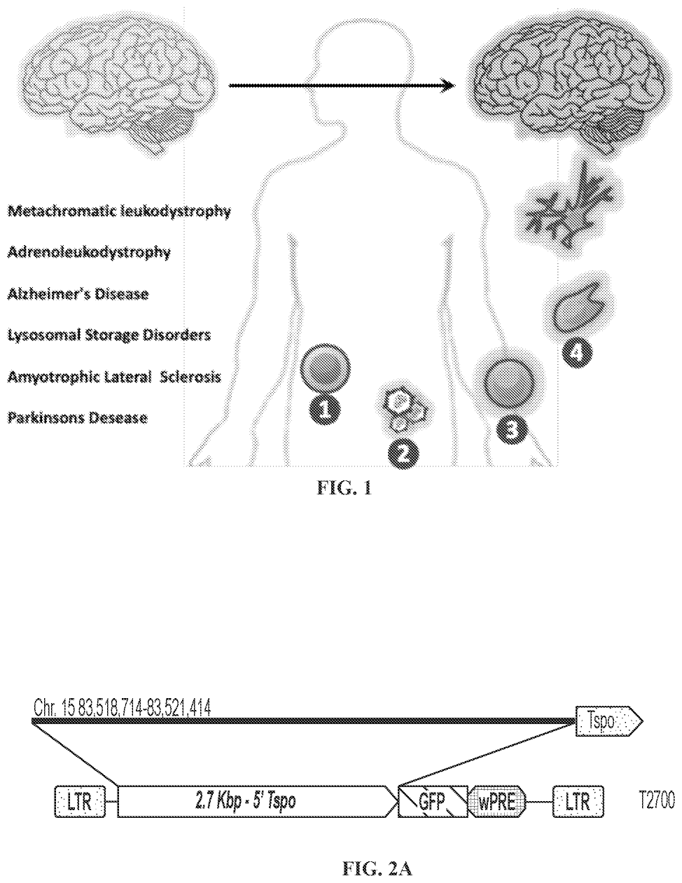

Efficient expression of therapeutic transgenes in cells residing in the central nervous system can potentially ameliorate, or even cure, neurodegenerative disease. Referring to FIG. 1, a mechanism is provided wherein human stem cells are obtained from a subject (1) and modified ex vivo to express a therapeutic transgene (2). The modified cells are then reinfused into a patient in need thereof (3), and the stem cells repopulate a central nervous system cell population (4). The invention features compositions and methods that are useful for expressing a transgene in a microglia cell. The present invention is based, at least in part, on the discovery that fragments of the murine promoter of the TSPO gene are capable of driving expression of a transgene in a microglial cell.

Not intending to be bound by theory, neuroinflammation is largely driven by microglial cell-activation and plays a major role in the pathogenesis and progression of Lysosomal Storage Disorders (LSD), among other neurodegenerative diseases. The beneficial effect of ex vivo gene therapy (GT) on lysosomal storage disorder (LSD) patients relies, at least in part, on the normalization of microglial cell homeostasis and reduction of neuroinflammation after the treatment. In some embodiments of a gene therapy (GT) for lysosomal storage disorders (LSDs), enzyme-competent microglial cells derived from genetically-modified hematopoietic stem cells (HSCs) engraft into a patient's Central Nervous System (CNS) where they release therapeutic factors to be available for all the resident CNS cells. Microglia-mediated delivery of immunomodulatory or other anti-oxidant factors could be used to mitigate neuroinflammation and neurodegeneration.

A suitable lentiviral vector for a gene therapy (GT) strategy to treat neurodegenerative diseases in various embodiments can drive regulated expression of the therapeutic transgene in hematopoietic stem progenitor cell-derived (HSPC-derived) microglial cells: upregulated in the presence of neuroinflammation and downregulated under homeostatic central nervous system (CNS) conditions. Such a transcriptional pattern is typical of the human translocator protein (TSPO) gene. Its overexpression is a hallmark of in vivo microglial activation in neurodegenerative disorders, suggesting TSPO promoter may be characterized by a minimal basal activity highly-inducible upon cell activation. The examples provided herein present an analysis used to identify regulatory elements that control this transcriptional pattern of a putative regulatory region of the human TSPO gene by integrating publicly-available transcriptional and epigenetic data produced by the Broad Institute-Encode Project in human hematopoietic cell lines (K562 cells). By a deep in silico analysis of the genomic region upstream and downstream to the human translocator protein (TSPO) transcriptional start site, regions were identified that were epigenetically marked as active promoters (H3K4me3⁺ and H3K27ac⁺) or active enhancers (H3K4me1⁺ and H3K27ac⁺) bound by hematopoietic-specific transcription factors as detected by Chip-seq genomic maps. Epigenetically-identified promoter regions were defined as actual promoters if active transcription was confirmed by RNA-seq data.

In the examples provided herein, four different putative regulatory elements were identified by this analysis (P1, P2, E1, and E2), which were subsequently modified for use in lentiviral vectors: small size, and removal of canonical and cryptic poly-adenylation and splicing signals. After these ad hoc modifications, putative regulatory elements were combined in different activation-responsive regulatory cassettes, which were cloned in five corresponding lentiviral vectors (LV MPP01-03-04-07-08) to control the expression of a Green Fluorescent Protein (GFP) reporter gene. These lentiviral vectors (LVs) were produced at a high titer and infectivity, and are currently under preclinical investigation in human microglial cells (HMC3 cell line) to test their transcriptional level under basal and cell-activation conditions. In various embodiments, the best vector(s) may be suitable for treatment of human neurodenerative diseases on account of their ability to safely and specifically induce a therapeutic level of transgene expression in pathological central nervous system (CNS) conditions.

Translocator protein (TPSO) is an outer mitochondrial membrane protein expressed in microglia cells and other phagocytic immune cells of the central nervous system. Expression of the TSPO gene is upregulated in microglia activated in response to pathological events and by lipopolysaccharide (LPS), a glycoprotein on the cell walls of Gram-negative bacteria (Visigalli et al. Neurobiol Dis 34, 51-62 (2009); Turner et al. Neurobiol Dis 15, 601-609 (2004), the contents of which are incorporated by reference in their entirety). The full-length TSPO promoter has been used to express transgenes in microglia, as described in PCT/US2017/056774, the contents of which are incorporated herein by reference in their entirety.

The full-length TSPO promoter is about 2700 nucleotides, which represents a significant percentage of the packaging capacity of most viral vectors. To compensate for the limited packaging capacity of viral vectors, some aspects of the present disclosure provide nucleic acid molecules comprising a truncated TSPO promoter operably linked to a coding sequence of a polynucleotide or polypeptide. The nucleic acid molecules are plasmids in some embodiments. Plasmids, such as cloning vectors, can be used to manufacture many copies of a transgene cassette that comprises a truncated TSPO promoter operably linked to a coding sequence. The plasmid may be an expression vector that can be used in an in vitro or in vivo environment to express the encoded transgene. In some embodiments, the nucleic acid molecules comprising a truncated TSPO promoter operably linked to a coding sequence of a polynucleotide or polypeptide is an isolated nucleic acid molecule.

In some embodiments, the truncated TSPO promoter comprises a nucleic acid having 100, 200, 300, 400, 500, 600, 700, 800, 900, 1000, 1100, 1200, 1300, 1400, 1500, 1600, 1700, 1800, 1900, 2000, 2100, 2200, 2300, or even 2400 fewer nucleotide than the wildtype TSPO promoter. For example, in some embodiments, the nucleic acid sequence of a truncated TSPO promoter consists of SEQ ID NO. 3 (T1600), SEQ ID NO: 5 (T1200), or SEQ ID NO: T300. In some embodiments, the truncated TSPO promoter is a non-naturally occurring nucleic acid sequence. In some embodiments, the non-naturally occurring nucleic acid sequence of the TSPO promoter is derived from the nucleic acid sequence of the human TSPO promoter. .

In some embodiments of the present disclosure, cultured cells are transfected with an expression vector comprising a transgene, wherein the transgene includes a truncated TSPO promoter operably linked to a coding sequence to express the encoded polynucleotide or polypeptide. To efficiently induce the cells to express the transgene's encoded polynucleotide or polypeptide, the cells are contacted with an activating agent, including but not limited to LPS. In some embodiments, the cells are stably transfected with a plasmid comprising the transgene as well as additional nucleic acid sequences that promote integration into a recipient cell's genome of a coding sequence of a transgene operably linked to a truncated TSPO promoter. In some embodiments, microglia cells, or precursors thereof, are transduced. In some embodiments, hematopoietic stem progenitor cells (HSPCs), or progeny thereof, are transduced. Methods of transfecting cells in a culture are well-known in the art.

In some embodiments of the present disclosure, cells may be transduced with a viral vector comprising a transgene operably linked to a truncated TSPO promoter is packaged within the vector. To transduce cells, viral vectors are assembled, and the nucleic acid molecule comprising a truncated TSPO promoter operably linked to a coding sequence are packaged within the vector. Because of the limited packaging capacity of viral vectors, the presently disclosed truncated TSPO promoter system has distinct advantages over previous systems using full-length promoters. By employing a truncated TSPO promoter, a larger coding sequence or additional regulatory sequences can be included in the transgene.

Viral vectors suitable for transducing microglia, or precursors thereof, or HSPCs, or progeny thereof, include but are not limited to lentiviral vectors, gamma-retroviral vectors, adenoviral vectors, and adeno-associated viral vectors. These vector systems are well-known in the art. In some embodiments, avian viral vectors are used to transduce microglia, or precursors thereof, or HSPCs, or progeny thereof. Avian viral vector systems include those described in U.S. Pat. No. 8,642,570, DE102009021592, PCT/EP2010/056757, and EP2430167, the contents of which are incorporated herein by reference in their entirety.

In some embodiments of the present disclosure, the transgene encodes a reporter polypeptide comprising detectable label. Examples of a reporter peptide include, but are not limited to, green fluorescent protein (GFP) and blue fluorescent protein (BFP). In some embodiments, expression of an expressed transgene is detected by contacting the cell with a detectably labeled reagent that specifically binds the expressed polynucleotide or polypeptide. In some embodiments, the reagent that specifically binds an expressed polypeptide is an antibody. Methods for producing antibodies and detectably labeled antibodies are known in the art. In some embodiments, the reagent that specifically binds an expressed nucleic acid is a detectably labeled polynucleotide comprising a nucleic acid sequence complementary to the expressed nucleic acid molecule. Detectable labels can be used to identify modified cells expressing the coding sequence of the transgene.

The present disclosure also includes vectors (e.g., recombinant plasmids) that include nucleic acid molecules (e.g., transgenes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include the nucleotide sequence comprising the TSPO promoter operably linked to a coding sequence. The vector may include additional regulatory sequences such as terminator sequences, long terminal repeats, untranslated regions, and the like. Recombinant expression vectors allow for expression of the genes or nucleic acids included in them.

Methods of introducing exogenous nucleic acid molecules into a cell (e.g., cells comprising a transgene having a truncated TSPO promoter) are known in the art. For example, eukaryotic cells can take up nucleic acid molecules from the environment via transfection (e.g., calcium phosphate-mediated transfection). “Stable transfection” refers to integration of the transfected nucleic acid into the recipient cell's genome, thereby allowing transmission of the transfected nucleic acid to the recipient cell's progeny. In some embodiments of the present disclosure, a nucleic acid molecule comprising a transgene as described herein comprises a nucleotide sequence that facilitates integration of the nucleic acid molecule into genome of a cell. Cells which have been stably transfected or transduced by the introduced DNA comprising a truncated TSPO promoter can be selected, for example, by including coding sequences in the vector for one or more markers that allow for selection of host cells which contain the expression vector. The markers can be a selective marker or a screenable marker.

In some embodiments, are modified via transduction. Transduction of most cell types can be accomplished with retroviral, lentiviral, adenoviral, adeno-associated, and avian virus systems, and such systems are well-known in the art. While retroviral systems are generally not compatible with neuronal cell transduction, lentiviruses are well-suited for transducing stem cells as well as neuronal cells. Thus, in some embodiments of the present disclosure, the viral vector system is a lentiviral system.

In some embodiments, the viral vector system is an avian virus system, for example, the avian viral vector system described in U.S. Pat. No. 8,642,570, DE102009021592, PCT/EP2010/056757, and EP2430167, the contents of which are incorporated herein by reference in their entirety. In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell. The components of a viral vector are encoded on plasmids, and because efficiencies of transduction decrease with large plasmid size, multiple plasmids that have different viral sequences necessary for packaging may be necessary. For example, in a lentiviral vector system, a first plasmid may comprise a nucleotide sequence encoding a Group antigens (gag) and/or a reverse transcriptase (pol) gene, while a second plasmid encodes regulator of expression of virion proteins (rev) and/or envelope (env) genes. The exogenous nucleic acid molecule comprising a transgene can be packaged into the vector and delivered into a recipient cell where the transgene is integrated into the recipient cell's genome. Additionally, the transgene may be packaged using a split-packaging system as described in U.S. Pat. No. 8,642,570, DE102009021592, PCT/EP2010/056757, and EP2430167.

In some embodiments, after the introduction of one or more vector(s), host cells are cultured prior to administration to a subject. Expression of recombinant proteins encoded in the vectors can be detected by immunoassays including Western blot analysis, immunoblot, and immunofluorescence.

### Hematopoietic Cell Transplantation

In some embodiments, a cell, such as HSPCs and/or their progeny, that comprise a transgene having a truncated TSPO promoter can serve as vehicles for therapeutic molecule delivery across the blood brain barrier by contributing to the turnover of myeloid cell populations in the brain. Methods for hematopoietic cell transplantation (HCT) and hematopoietic stem cell (HSC)-based gene therapy are known in the art and have been used to treat patients affected by non-hematological and non-oncological diseases affecting the nervous system, such as peroxisomal disorders and lysosomal storage diseases (LSDs) (Cartier et al. Science 326, 818-823 (2009); Biffi et al. Science 341, 1233158 (2013); Sessa et al. Lancet 388, 476-487 (2016)) and neurological diseases (Simard et al. Neuron 49, 489-502 (2006)). Indeed, HSPCs and/or their progeny can contribute to the turnover of myeloid cell populations in the brain (Ajami et al. Nat Neurosci 10, 1538-1543 (2007); Ajami et al. Nat Neurosci 14, 1142-1149 (2011); Biffi et al. J. Clin. Invest. 116, 3070-3082 (2006); Mildner et al. Nat Neurosci 10, 1544-1553 (2007); Capotondo et al. Proc Natl Acad Sci USA. 109, 15018-15023 (2012). Microglia's role in the progression and outcomes of these disorders has been described (Jeyakumar et al. Brain 126, 974-987 (2003); Wada et al. Proc Natl Acad Sci USA 97, 10954-10959 (2000); Ohmi et al. Proc. Natl. Acad. Sci. USA 100, 1902-1907 (2003); Eichler et al. Ann Neurol 63, 729-742 (2008), the contents of each are incorporated herein in their entirety).

Transplant efficiency can be improved with an ablative preconditioning regimen to destroy endogenous microglia, such as that described in International Application No. PCT/US2017/056774, the contents of which are incorporated herein by reference in their entirety. In some embodiments of the present disclosure, the ablative conditioning regimen comprises administering an alkylating agent to a subject prior to transplantation. In some embodiments, the alkylating agent is busulfan. Busulfan is capable of ablating functionally-defined brain-resident microglia precursors (Capotondo et al. Proc Natl Acad Sci USA. 109, 15018-15023 (2012); Wilkinson et al. Mol Ther 21, 868-876 (2013)). In addition to alkylation agents, such as busulfan, CSF-1R inhibitors (e.g., PLX3397 and PLX5622), and liposomal clodronate may be used (Han et al. Molecular Brain, 10:25, 2017), optionally they may be used in combination with the nanoparticles described, for example, in WO2019191650, which is incorporated herein.

In general, the term nanoparticle refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs.

Such nanoscale particles are used in biomedical applications as drug carriers or imaging agents and may be adapted for similar purposes in the present invention. Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants. In one embodiment, nanoparticles based on self-assembling bioadhesive polymers are contemplated, which may be applied to oral delivery of agents, intravenous delivery of agents and nasal delivery of agents, all to the brain. Other embodiments, such as oral absorption and ocular deliver of hydrophobic drugs are also contemplated. The molecular envelope technology involves an engineered polymer envelope which is protected and delivered to the site of the disease (Mazza et al. ACS Nano 7, 1016-1026 (2013); Siew et al. Mol Pharm 9, 14-28 (2012); Lalatsa et al. J Control Release 161, 523-536 (2012); Lalatsa et al. Mol Pharm 9, 1665-1680 (2012); Garrett et al. J Biophotonics 5, 458-468 (2012); Uchegbu, Expert Opin Drug Deliv 3, 629-640 (2006); Uchegbu et al. Int J Pharm 224, 185-199 (2001); Qu et al. Biomacromolecules 7, 3452-3459 (2006)).

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation, which includes a particle in accordance with the present invention. A particle in accordance with the present invention is any entity having a greatest dimension (e.g. diameter) of less than 100 microns (μμm). In some embodiments, inventive particles have a greatest dimension of less than 10 p.m. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS).

Particle delivery systems within the scope of the present invention may be provided in any form, including but not limited to solid, semi-solid, emulsion, or colloidal particles. As such any of the delivery systems described herein may be provided as particle delivery systems within the scope of the present invention.

Despite microglia having a developmental origin distinct from that of bone marrow-derived myelomonocytes (Ginhoux et al. Science 330, 841-845 (2010), the contents of which are incorporated herein by reference in their entirety), it has recently been demonstrated that under specific experimental conditions, cells of donor origin showing a microglia-like phenotype and expressing some microglia surface markers could be successfully generated in the brain of mice transplanted with donor HSPCs. HSPCs have the capacity to generate new populations of myeloid and microglia cells that can exert therapeutic effects in the central nervous system (CNS). This disclosure provides compositions comprising cells modified to have a transgene comprising a truncated TSPO promoter and enhanced methods for engrafting such cells.

Transplantating HSPCs or their progeny generates transcriptionally-dependable microglia through a stepwise process similar to physiological post-natal microglia maturation. Cells comprising a transgene having a truncated TSPO promoter are able to generate new microglia upon transplantation into myeloablated recipients are retained within human and murine long-term hematopoietic stem cells (HSCs). In some embodiments, microglia cells can be generated after intracerebroventricular delivery of cells (e.g., HSPCs) comprising a transgene having a truncated TSPO promoter, which unexpectedly results in faster and more widespread microglia replacement compared to delivery of wild-type cells.

### Pharmaceutical Compositions

Compositions contemplated in the present disclosure include pharmaceutical compositions comprising cells that are modified to have a transgene comprising a truncated TSPO promoter. In some embodiments, the transgene encodes a therapeutic polypeptide, and the truncated TSPO promoter drives expression of the polypeptide. In some embodiments, the therapeutic polypeptide is neuroprotective or is required to replace a missing metabolic enzyme. The pharmaceutical compositions contemplated herein can comprise autogenic or allogenic cells. In some embodiments the cells are HSPCs comprising a transgene having a truncated TSPO promoter.

The cells comprising a transgene having a truncated TSPO promoter as described herein can be administered as therapeutic compositions (e.g., as pharmaceutical compositions). Cellular compositions as described herein can be provided as sterile liquid preparations, e.g., isotonic aqueous solutions, suspensions, emulsions, dispersions, or viscous compositions, which may be buffered to a selected pH. A liquid preparation may be easier to prepare than a gel, another viscous composition, and a solid composition. Additionally, a liquid composition may be more convenient to administer (i.e., by injection). Viscous compositions, on the other hand, can be formulated within the appropriate viscosity range to provide longer contact periods with specific tissues. Liquid or viscous compositions can comprise a carrier, which can be a solvent or dispersing medium comprising, for example, water, saline, phosphate buffered saline, polyol (e.g., glycerol, propylene glycol, liquid polyethylene glycol, and the like), and suitable mixtures thereof.

Sterile injectable solutions can be prepared by incorporating the cells described herein in a sufficient amount of an appropriate diluent. Such compositions may be in admixture with a suitable carrier or excipient such as sterile water, physiological saline, glucose, dextrose, or another carrier or excipient suitable for delivering live cells to a subject. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation.

Additives that enhance the stability and sterility of the cellular compositions, including antimicrobial preservatives, antioxidants, chelating agents, and buffers, can be added. Prevention of the action of microorganisms can be ensured by an antibacterial or antifungal agent including, but not limited to, parabens, chlorobutanol, phenol, and sorbic acid. According to the present disclosure, however, any vehicle, diluent, or additive used must be compatible with the cells.

The compositions can be isotonic, i.e., they have the same osmotic pressure as blood and cerebrospinal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride may be suitable for buffers containing sodium ions.

Viscosity of the compositions, if desired, can be maintained at a selected level using a pharmaceutically acceptable thickening agent. In some embodiments, the thickening agent is methylcellulose, which is readily and economically available and is easy to work with. Other suitable thickening agents include, but are not limited to, xanthan gum, carboxymethyl cellulose, hydroxypropyl cellulose, and carbomer. The concentration of the thickener will depend upon the agent selected and the amount of the agent used. Suitable carriers and other additives may be chosen depending on the route of administration and the nature of the dosage form (e.g., a liquid dosage form can be formulated into a solution, a suspension, a gel, or another liquid form, such as a time release formulation or liquid-filled form).

An effective amount of cells to be administered can vary for the subject being treated. In one embodiment, between about 10⁴ to about 10⁸ cells, and in another embodiment between about 10⁵ to about 10⁷ cells are administered to a subject.

The skilled artisan can readily determine the amounts of cells comprising a transgene having a truncated TSPO promoter and optional additives, vehicles, and/or carrier in compositions to be administered. In one embodiment any additive (in addition to the cell(s)) is present in an amount of about 0.001% to about 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD5o in a suitable animal model e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

### Methods of Treatment

The present disclosure provides methods of treatment for a subject in need thereof by administering a cell comprising a transgene having a truncated TSPO promoter operably linked to a coding sequence, or a pharmaceutical composition comprising the cell, to the subject. In some embodiments, the subject in need of treatment has or is suspected of having a metabolic or neurological disease.

A health care professional may diagnose a subject as having a metabolic or neurological disease by the assessment of one or more symptoms of disease in the subject. The present disclosure provides methods of treating a metabolic or neurological disease or symptoms thereof that comprise administering to a subject (e.g., a mammal, such as a human) a therapeutically effective amount of a cell comprising a transgene having a truncated TSPO promoter drives expression of a therapeutic polynucleotide or polypeptide. In some embodiments, the cell is an HSPC or its progeny. In some embodiments, the cell is a microglial progenitor cell. In some embodiments, the cell is a microglia cell. Thus, the method in some embodiments comprises administering to the subject a therapeutically effective amount of a cell described herein sufficient to treat a metabolic or neurological disease or symptom thereof, under such conditions that the disease is treated.

The methods herein include administering to the subject (including a subject identified as in need of such treatment) an effective amount of cells described herein, or a composition comprising such cells as described herein to produce such effect. Such treatment will be suitably administered to a subject, particularly a human, suffering from, having, susceptible to, or at risk for, a metabolic or neurological disease, or a symptom thereof. In some embodiments, the methods herein include administering to the subject (including a subject identified as in need of such treatment) an effective amount of a compound described herein, or a composition described herein to produce such effect.

In some embodiments, the cell or the composition comprising the cell is administered to a subject in a targeted manner. For example, in some embodiments, a composition comprising a cell having a transgene comprising a truncated TSPO promoter is administered directly to a subject's brain. In some embodiments, the composition is delivered directly to the brain via intracerebroventricular administration. In some embodiments, the composition is delivered in this manner to the lateral ventricles of the subject's brain. Methods of administration useful in the present disclosure are described, for example in International Application No. PCT/US2020/045106, which is incorporated by reference in its entirety.

Alternatively, the composition may be delivered systemically, such as by intravenous administration. Cells administered in such a manner must traverse the blood brain barrier prior to engrafting in the subject's brain. Other modes of administration (parenteral, mucosal, implant, intraperitoneal, intradermal, transdermal, intramuscular, intracerebroventricular injection, intravenous including infusion and/or bolus injection, and subcutaneous) are generally known in the art. In some embodiments, cells are administered in a medium suitable for injection, such as phosphate buffered saline, into a subject. Because the cells being administered to the subject are intended to repopulate microglial cells, intracerebroventricular administration may be advantageous to routes that require crossing the blood brain barrier.

In some embodiments, the transplanted cells are meant to replace endogenous cells (i.e., microglial cells); therefore, methods of treating a subject having, susceptible to, or at risk of developing a metabolic or neurological disease further comprise administering to a subject an agent for ablating endogenous cells, such as microglia prior to administering a cell comprising a transgene encoding a therapeutic polypeptide and having a truncated TSPO promoter. In some embodiments, the agent is an alkylating agent. In some embodiments, the alkylating agent is busulfan. In some embodiments, nanoparticle delivery of alkylating agents may be effective in creating a suitable environment for engraftment of transplanted cells, as described in International Application No. PCT/US2017/056774, the contents of which are incorporated herein by reference in their entirety.

### Kits

The present disclosure contemplates kits for the treatment or prevention of a metabolic or neurological disease. In some embodiments, the kit comprises a composition comprising a cell that has a transgene comprising a truncated TSPO promoter. In some embodiments, the cell comprising a transgene having a truncated TSPO promoter is modified to express a therapeutic polypeptide. The kit can include instructions for a treatment protocol, reagents, equipment (test tubes, reaction vessels, needles, syringes, etc.), and standards for calibrating or conducting the treatment protocol. The instructions provided in a kit according to the present disclosure may be directed to suitable operational parameters in the form of a detectable label or a separate insert. Optionally, the kit may further comprise a standard or control information so that the test sample can be compared with the control information standard to determine if a consistent result is achieved. In some embodiments, the kit includes a nanoparticle for ablative conditioning of endogenous microglial cells.

In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a composition of the invention is provided together with instructions for administering the composition to a subject having or at risk of developing a metabolic or neurological disease or disorder of the central nervous system. The instructions will generally include information about the use of the composition for the treatment or prevention of the disease or disorder. In other embodiments, the instructions include at least one of the following: description of the therapeutic composition; dosage schedule and administration for treatment or prevention of a neurological disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Murine TSPO Promoter Cloning and Testing: Proof of Concept Study

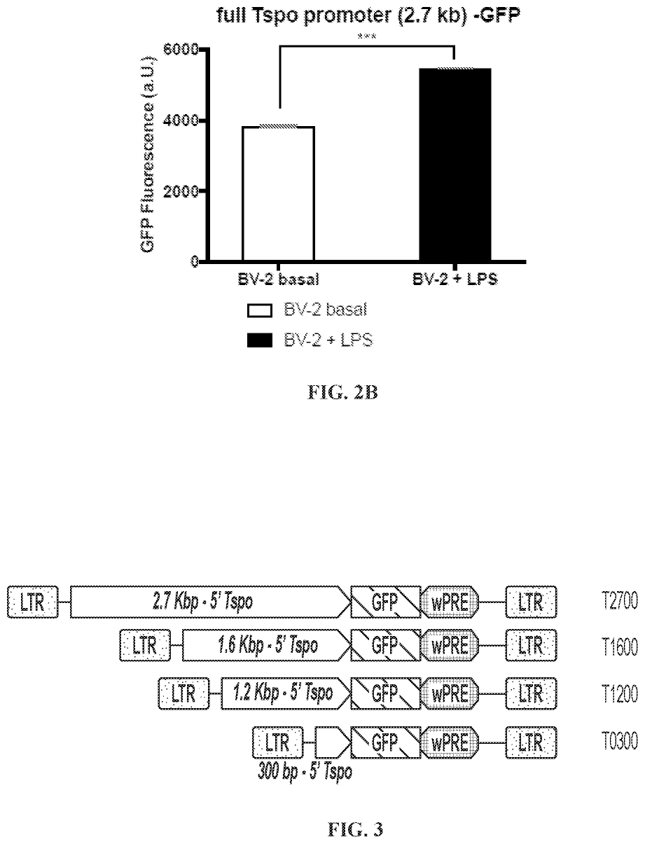

Cells transduced via a lentiviral vector comprising a nucleic acid molecule having the full-length mTspo promoter operably linked to a coding sequence for GFP were able to express the reporter gene. A total of 2.7 kb of genomic DNA from mouse genomic DNA containing the promoter sequence 5′ of the murine TSPO gene were PCR cloned into T2700, a third-generation lentiviral transfer plasmid. The vector contains a cassette encoding GFP and the Woodchuck hepatitis virus post-transcriptional regulatory element (Wpre) (FIG. 2A). In vivo LPS administration into the brain triggers TSPO overexpression in microglia cells. To assess the ability of the full-length TSPO promoter to drive expression of the encoded GFP, cells were administered liposaccharide (LPS), a well-known inducer of inflammation in vivo in a murine microglia cell line, BV-2 cells, and in brain resident microglia. LPS challenge of the transduced cells resulted in GFP expression (FIG. 2B).

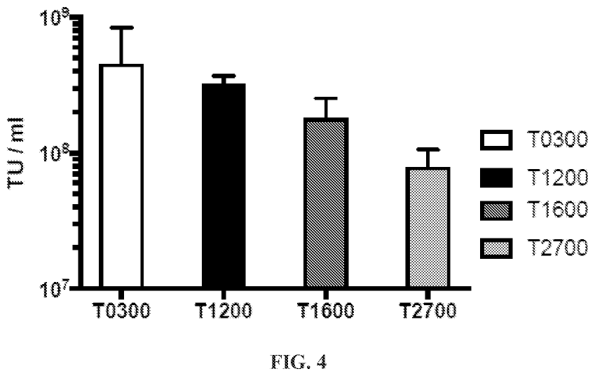

One limitation for a broader use of native promoter sequences in lentiviral vectors is the cargo limitations of vector capsids. To overcome this problem, fragments of the mTspo 5′ UTR were cloned and tested for promoter activity. The following novel transfer plasmids were generated: T1600, T1200, and T300 containing approximately 1.6 kb, 1.2 kb, and 300 base pairs of the mTspo 5′ UTR, respectively (FIG. 3). These promoter fragments are referred to by the plasmid name in which they cloned. For example, the T1600 promoter refers to the promoter fragment in the T1600 transfer plasmid. Lentiviral vectors were generated by co-transfection of the new constructs with third-generation helper plasmids in HEK293T cells. The constructs comprising shorter promoters were more proficiently assembled into recombinant vectors as evidenced by their higher titers yielded as compared to the low titer obtained with T2700 (FIG. 4).

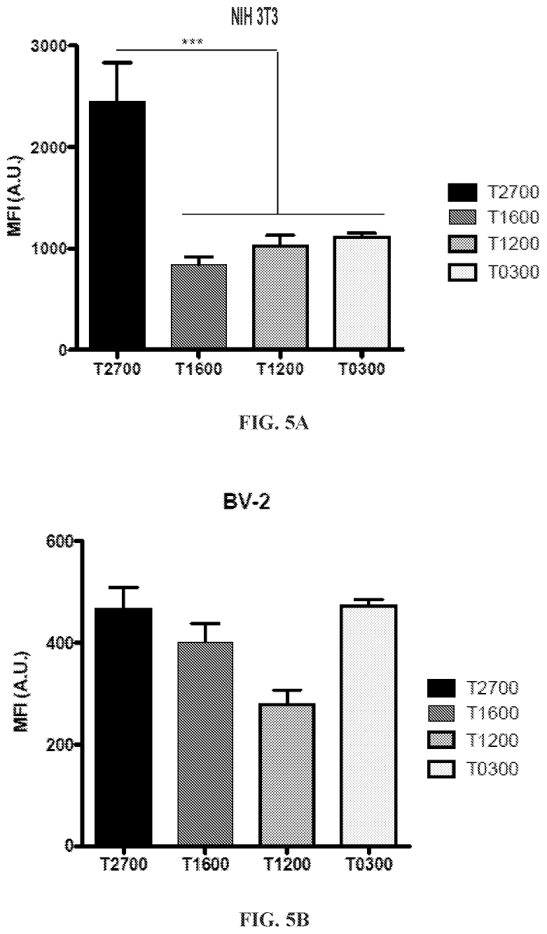

Lentiviral vector supernatants from the HEK293T cultures were used to test the infectivity of the vectors in several cell lines: human fibroblast, HEK293T; mouse microglia, BV-2; and mouse fibroblast, NIH3T3. Ectopic GFP expression driven by the mTspo promoter in mouse fibroblasts depended greatly on the distal 1 kb in the 5′ region of the full promoter, but eliminating this terminal sequence did not have a drastic effect on GFP expression in microglia cells under basal conditions (FIGS. 5A and 5B).

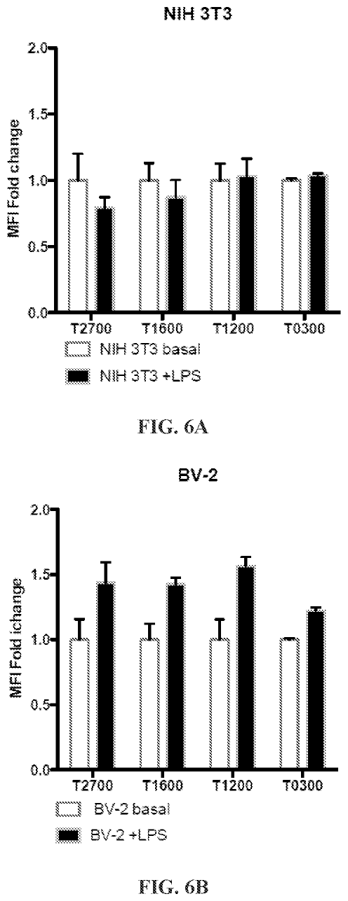

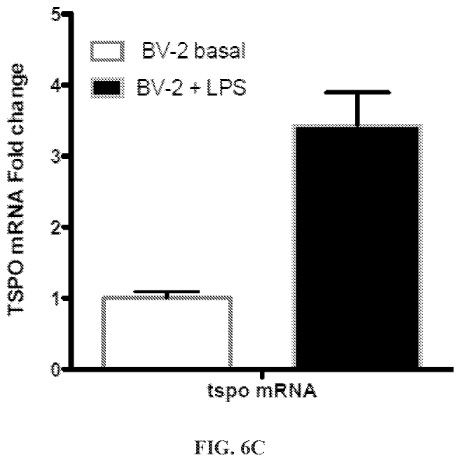

Using pools of transduced cells, the effect of LPS (1 μg/ml) on GFP expression was tested. A change in GFP intensity was observed only when BV-2 microglia cells were challenged with LPS, while LPS did not affect GFP expression driven by any of the mTspo vectors assayed in NIH3T3 fibroblasts (FIGS. 6A and 6B). In BV-2 cells, in addition to the full mTspo promoter, all fragments, including the minimal T300 promoter, were stimulated by LPS exposure (FIG. 6C). Although modest compared to TSPO induction in vivo, the observed induction is within a range comparable to mTspo mRNA changes measured in BV-2 microglia cells upon LPS stimulation. The mTspo reporter lentiviral vectors were used to deliver transgenes into microglia in vivo.

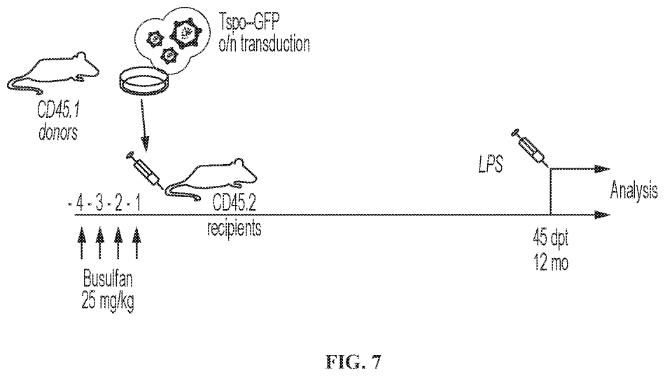

A series of transplantations into C57/BL mice took advantage of the CD45.1-CD45.2 mismatch system (FIG. 7). The recipient mice were conditioned with a cumulative dose of 100 mg/kg of busulfan administered in four doses spaced 24 hours apart. Cells were harvested from tibias, femur, hips, and humerus of CD45.1 mice the day before the transplant. Bone marrow cells underwent lineage depletion and the resulting cell suspension was transduced overnight at a multiplicity of infection (MOI) of 100 TU/cell in StemSpan supplemented with cytokines (i.e., mIL-3, mFLt-3, mIL-6, mSCF). The following morning, cells were washed in phosphate buffered saline (PBS), counted and resuspended for intracerebroventricular (ICV) administration. 3×10⁵ cells were injected in the right cerebral ventricle in a volume of 5 μl. Recipient mice received an infusion of 2×10⁶ CD45.2 total BM support cells four days after the ICV injection to rescue them from aplasia.

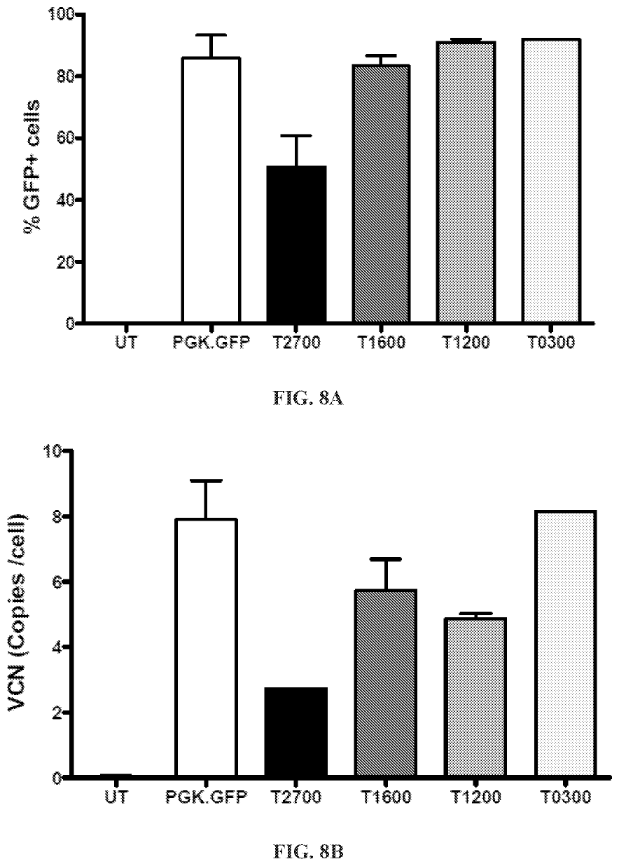

The full length mTspo promoter, T1600, T1200, T300, and a non-inducible PGK-GFP control were tested (Table 1). To maximize responsiveness of the murine TSPO promoters, the transplanted mice in each cohort were split into two cohorts: one to assess basal GFP expression and microglia reconstitution, and the other was challenged with LPS one day before sacrifice. The longest murine TSPO promoters achieved modest levels of labelling of HSPCs (FIGS. 8A and 8B).

During the active process of expansion and differentiation of the transplanted cell progeny, the majority of microglia are transiently activated (Tap), and these cells have an increased level of murine TSPO expression, which complicates evaluation of the impact of TLR4 activation. For this reason, shortly after transplant, the animals were divided into two timepoints of analysis. A small fraction of the transplanted animals at 45 days after transplant were analyzed, but the majority of the animals were evaluated at six months post-transplant.

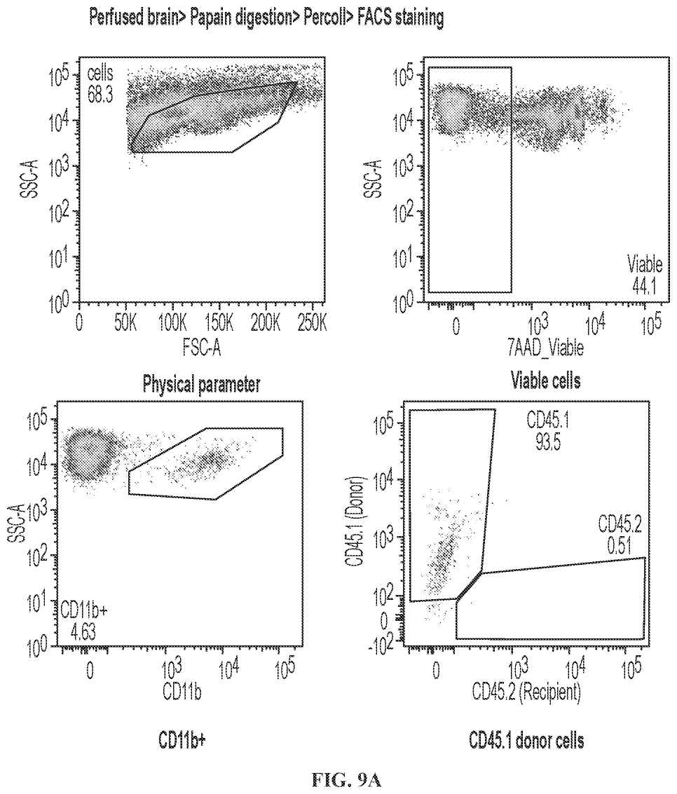

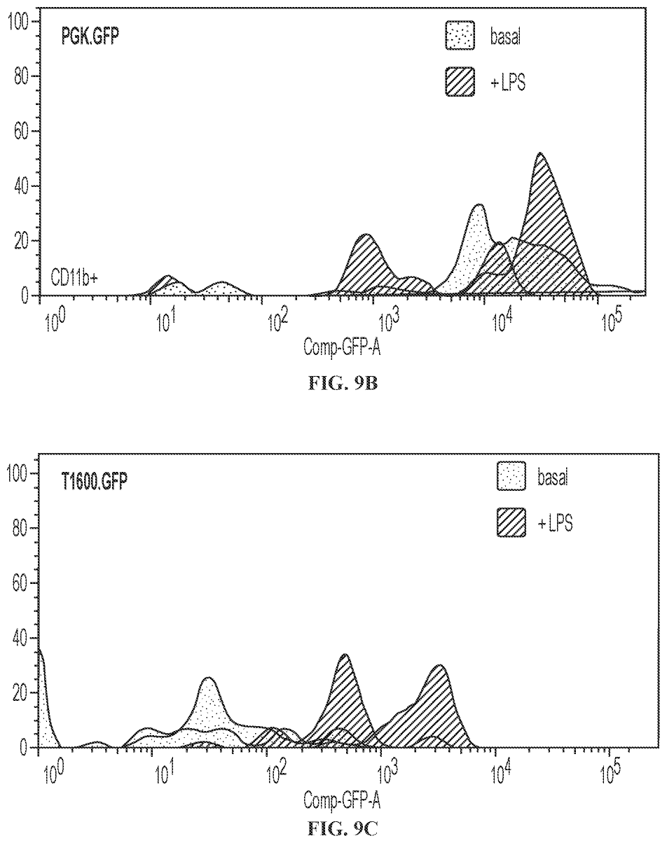

Upon perfusion with saline, brains, bone marrow, and spleen were collected and processed for flow cytometry. Analysis confirmed induction of GFP expression in donor-derived microglia-like cells (CD45⁺CD11b⁺) cells upon LPS stimulation (FIGS. 9A-9C). Of note, basal expression driven by murine TSPO promoter variants was lower than that driven by the control PGK promoter, but the levels of expression achieved upon stimulation by LPS of the murine TSPO promoter variants were comparable to the PGK control.

TABLE 1

| | Lin- | | | | | | | |
|---|---|---|---|---|---|---|---|---|
| Group | donor | Transduction | | MOI | Recipient | Conditioning | Dose | Treatment |
| A | CD45.1 | full-Tspo | (2700 bp) | 100 | CD45.2 | Busulfan | 4 × 25 mg/kg | n/a |
| | | | | | | | | LPS 24 h before sac |
| B | CD45.1 | PGK | (700 bp) | 100 | CD45.2 | Busulfan | 4 × 25 mg/kg | n/a |
| | | | | | | | | LPS 24 h before sac |
| C | CD45.1 | 1600 | bp Tspo | 100 | CD45.2 | Busulfan | 4 × 25 mg/kg | n/a |
| | | | | | | | | LPS 24 h before sac |
| D | CD45.1 | 1200 | bp Tspo | 100 | CD45.2 | Busulfan | 4 × 25 mg/kg | n/a |
| | | | | | | | | LPS 24 h before sac |
| E | CD45.1 | 300 | bp Tspo | 100 | CD45.2 | Busulfan | 4 × 25 mg/kg | n/a |
| | | | | | | | | LPS 24 h before sac |

### Example 2: Human TSPO Promoter Cloning and Testing for Clinical Development

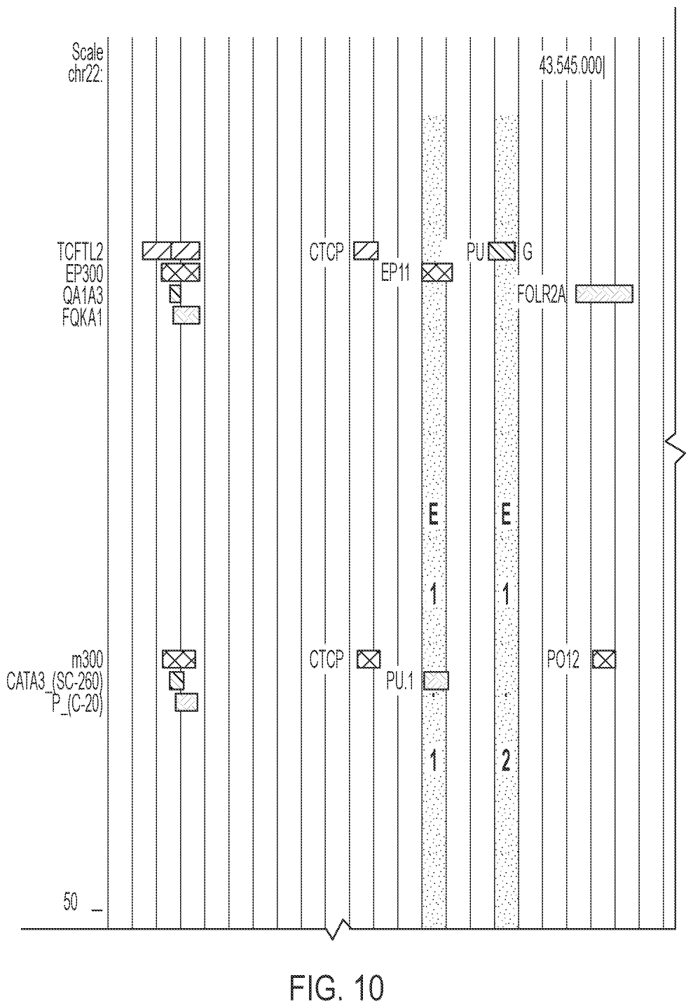

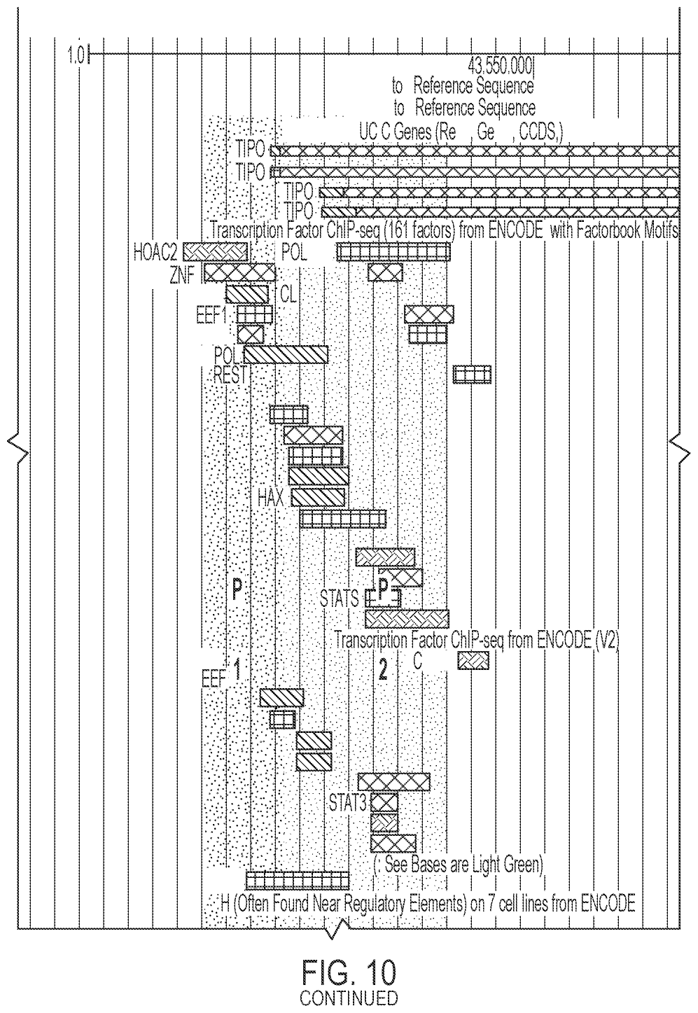

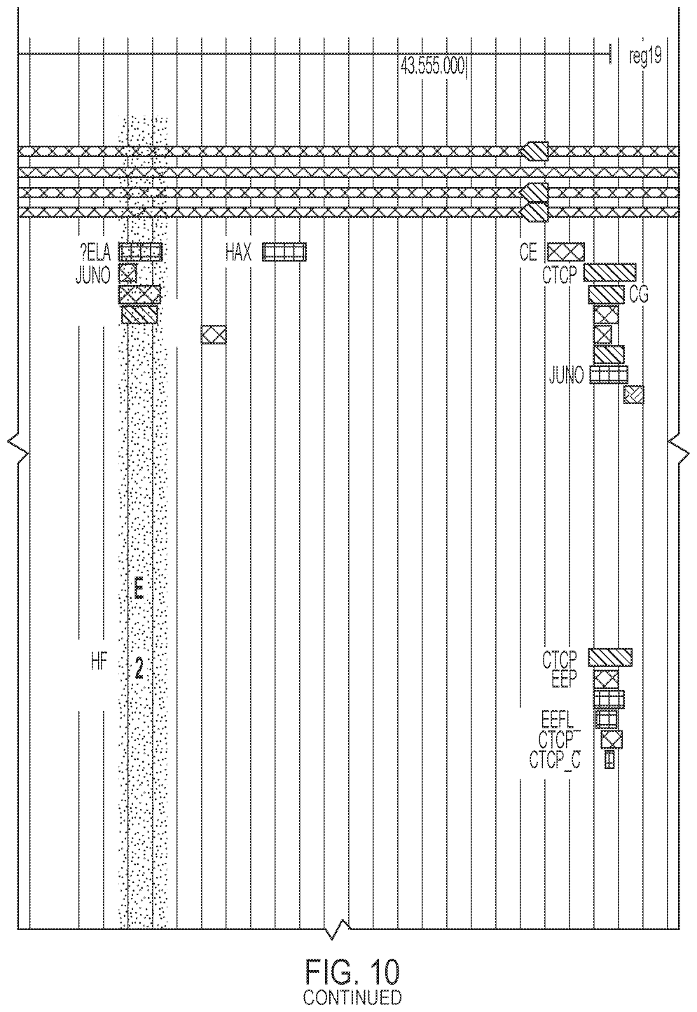

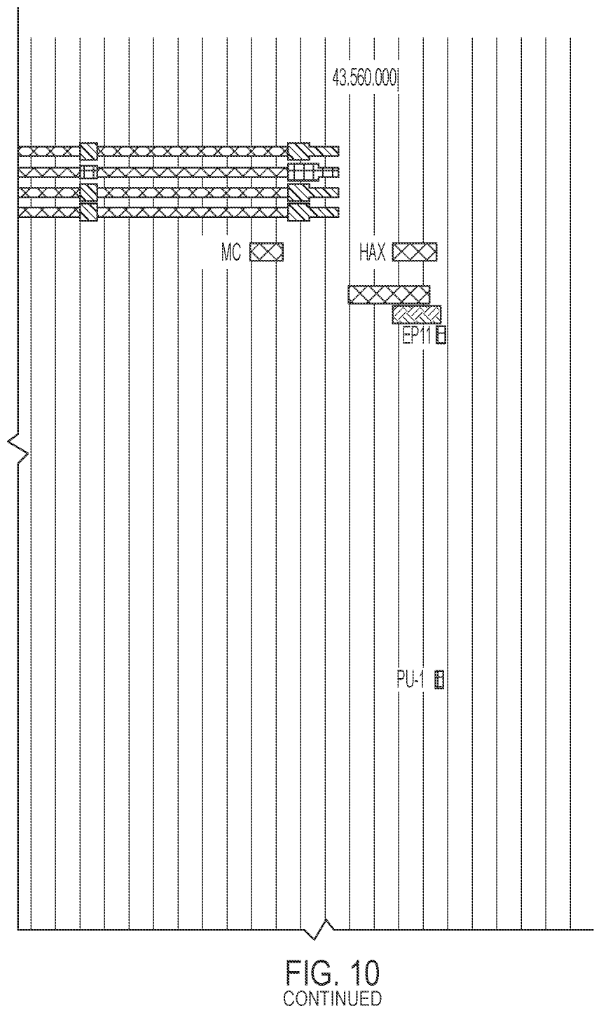

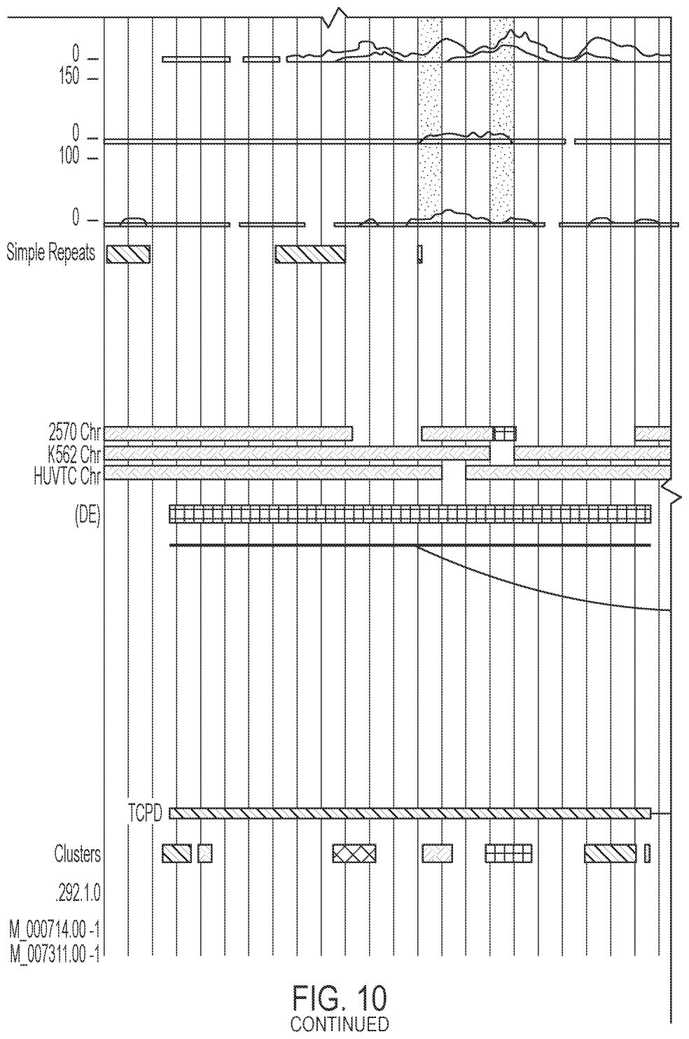

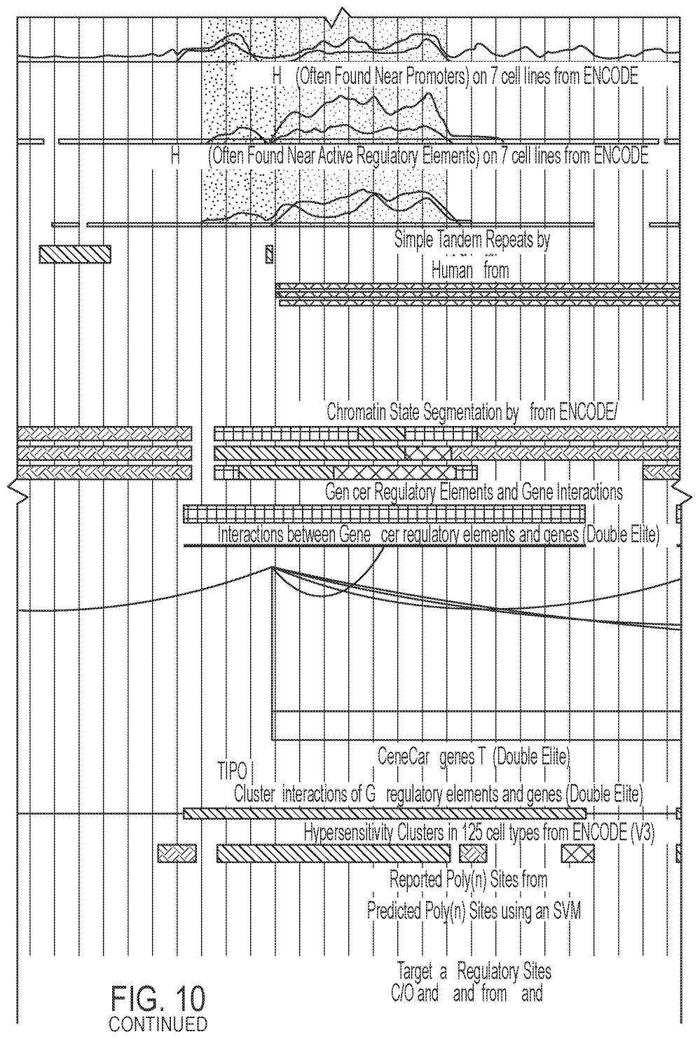

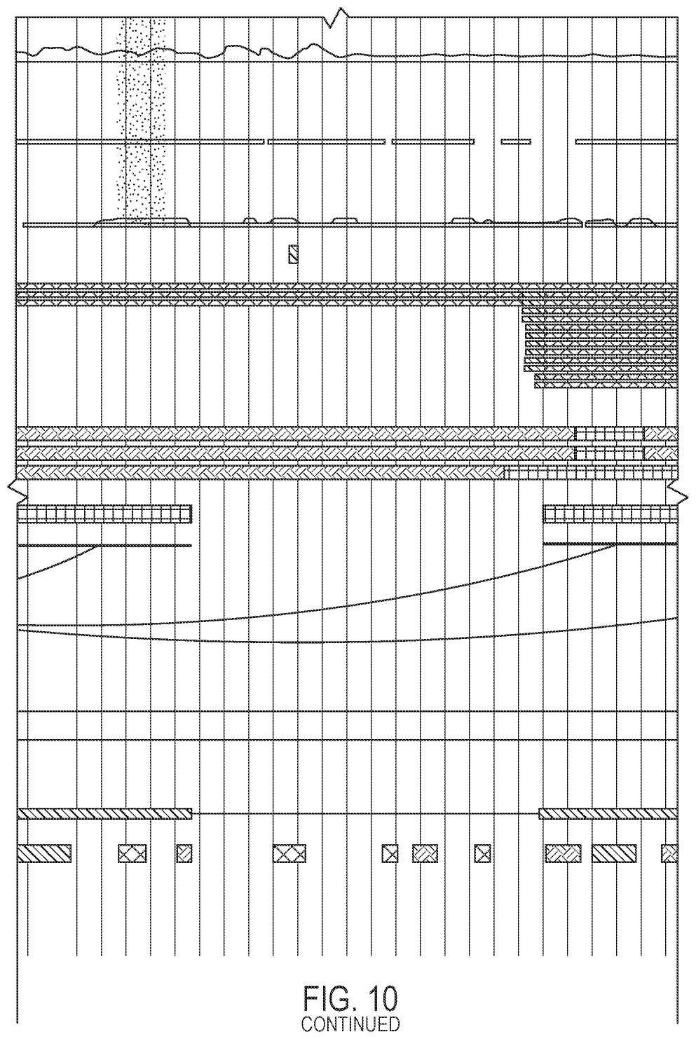

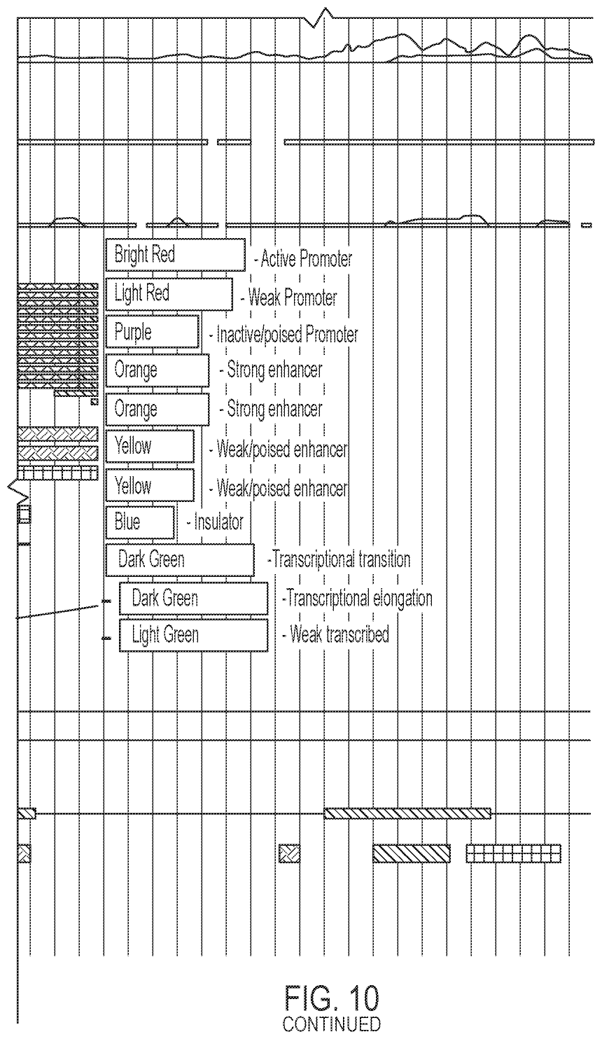

Based on the positive findings observed using the murine TSPO promoter sequences in a homologous setting, use of the human TSPO promoter sequence to drive microglia specific and ambient responsive expression of therapeutic transgenes was investigated. Despite the large amount of human genomic information and the extensive use of TSPO radioligands to follow patients undergoing neurodegenerative disease progression, the characterization of the human promoter driving TSPO expression has not been thoroughly addressed. Mouse and human TSPO sequences are in genomic loci with very poor homology, and because the human TSPO putative promoter region extends over several kilobases on chromosome 22, it is not suitable to use as such in a lentiviral vector for comparative analyses. Hence, synthetic promoters derived from the human sequence were generated that were bioinformatically predicted to control human TSPO expression. Special interest was directed to those synthetic promoters regulating TSPO expression in microglia-related tissues found in the ENCODE and other databases (FIG. 10).

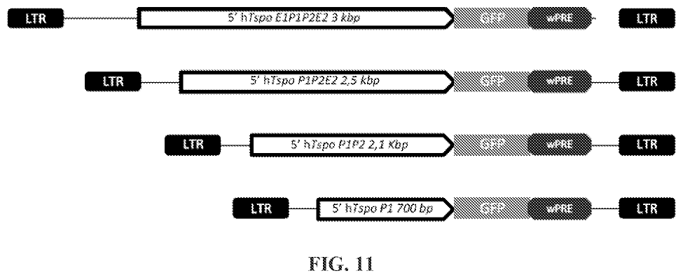

To conserve as much of the genuine specificity established by the union of transcription factors, while keeping a reduced size suitable for use in viral vectors, the following promoter sequences were cloned:
i) The P1 5′ promoter
ii) The extended P1+P2 promoter
iii) The synthetic concatenation of P1, P2 and the intronic enhancer E2
iv) The synthetic union of E1+P1+P2+E2
   These promoter sequences were cloned into the same lentiviral vector transfer plasmid employed for murine TSPO (FIG. 11). These transfer plasmids are used for lentiviral vector production runs. In vitro characterization of the novel human TSPO promoter variants in the lentiviral vectors constructs allows determination of the packaging capacity of the vectors. The functionality of the promoter variants is assessed by GFP expression in basal conditions in different cell lines, including human microglia cells as described above.

Constructs are selected for further expression characterization using LPS or other TLR-4 agonist responsiveness in relevant cell lines. Additionally, in vivo characterization of the most promising constructs is studied in a humanized model of microglia reconstitution in NSG animals. In vivo exploratory assessment of responsiveness of the promoter(s) in a mouse-into-mouse setting can be assessed.

Sequence of lentiviral vector:

pCCLsin.PPT.hPGK.GFP.Wpre_mut_AMP
caggtggcacttttcggggaaatgtgcgcggaacccctatttgtttatttttctaaatacattcaaatatgtatccgctcatgagacaataaccctgata
aatgcttcaataatattgaaaaaggaagagtatgagtattcaacatttccgtgtcgcccttattcccttttttgcggcattttgccttcctgtttttgctcac
ccagaaacgctggtgaaagtaaaagatgctgaagatcagttgggtgcacgagtgggttacatcgaactggatctcaacagcggtaagatccttg
agagttttcgccccgaagaacgttttccaatgatgagcacttttaaagttctgctatgtggcgcggtattatcccgtattgacgccgggcaagagca
actcggtcgccgcatacactattctcagaatgacttggttgagtactcaccagtcacagaaaagcatcttacggatggcatgacagtaagagaatt
atgcagtgctgccataaccatgagtgataacactgcggccaacttacttctgacaacgatcggaggaccgaaggagctaaccgcttttttgcaca
acatgggggatcatgtaactcgccttgatcgttgggaaccggagctgaatgaagccataccaaacgacgagcgtgacaccacgatgcctgtag
caatggcaacaacgttgcgcaaactattaactggcgaactacttactctagcttcccggcaacaattaatagactggatggaggcggataaagtt
gcaggaccacttctgcgctcggcccttccggctggctggtttattgctgataaatctggagccggtgagcgtgggtctcgcggtatcattgcagc
actggggccagatggtaagccctcccgtatcgtagttatctacacgacggggagtcaggcaactatggatgaacgaaatagacagatcgctga
gataggtgcctcactgattaagcattggtaactgtcagaccaagtttactcatatatactttagattgatttaaaacttcatttttaatttaaaaggatcta
ggtgaagatcctttttgataatctcatgaccaaaatcccttaacgtgagttttcgttccactgagcgtcagaccccgtagaaaagatcaaaggatctt
cttgagatcctttttttctgcgcgtaatctgctgcttgcaaacaaaaaaaccaccgctaccagcggtggtttgtttgccggatcaagagctaccaact
ctttttccgaaggtaactggcttcagcagagcgcagataccaaatactgtccttctagtgtagccgtagttaggccaccacttcaagaactctgtag
caccgcctacatacctcgctctgctaatcctgttaccagtggctgctgccagtggcgataagtcgtgtcttaccgggttggactcaagacgatagtt
accggataaggcgcagcggtcgggctgaacggggggttcgtgcacacagcccagcttggagcgaacgacctacaccgaactgagataccta
cagcgtgagctatgagaaagcgccacgcttcccgaagggagaaaggcggacaggtatccggtaagcggcagggtcggaacaggagagcg
cacgagggagcttccagggggaaacgcctggtatctttatagtcctgtcgggtttcgccacctctgacttgagcgtcgatttttgtgatgctcgtca
ggggggcggagcctatggaaaaacgccagcaacgcggcctttttacggttcctggccttttgctggccttttgctcacatgttctttcctgcgttatc
ccctgattctgtggataaccgtattaccgcctttgagtgagctgataccgctcgccgcagccgaacgaccgagcgcagcgagtcagtgagcga
ggaagcggaagagcgcccaatacgcaaaccgcctctccccgcgcgttggccgattcattaatgcagctggcacgacaggtttcccgactgga
aagcgggcagtgagcgcaacgcaattaatgtgagttagctcactcattaggcaccccaggctttacactttatgcttccggctcgtatgttgtgtgg
aattgtgagcggataacaatttcacacaggaaacagctatgaccatgattacgccaagcgcgcaattaaccctcactaaagggaacaaaagctg
gagctgcaagcttggccattgcatacgttgtatccatatcataatatgtacatttatattggctcatgtccaacattaccgccatgttgacattgattatt
gactagttattaatagtaatcaattacggggtcattagttcatagcccatatatggagttccgcgttacataacttacggtaaatggcccgcctggct
gaccgcccaacgacccccgcccattgacgtcaataatgacgtatgttcccatagtaacgccaatagggactttccattgacgtcaatgggtggag
tatttacggtaaactgcccacttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttggcaccaaaatcaacggg
actttccaaaatgtcgtaacaactccgccccattgacgcaaatgggcggtaggcgtgtacggtgggaggtctatataagcagagctcgtttagtg
aaccggggtctctctggttagaccagatctgagcctgggagctctctggctaactagggaacccactgcttaagcctcaataaagcttgccttga
gtgcttcaagtagtgtgtgcccgtctgttgtgtgactctggtaactagagatccctcagacccttttagtcagtgtggaaaatctctagcagtggcgc
ccgaacagggacctgaaagcgaaagggaaaccagagctctctcgacgcaggactcggcttgctgaagcgcgcacggcaagaggcgaggg
gcggcgactggtgagtacgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgggcaagcagggagctagaa
cgattcgcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaagatagagg
aagagcaaaacaaaagtaagaccaccgcacagcaagcggccgctgatcttcagacctggaggaggagatatgagggacaattggagaagtg
aattatataaatataaagtagtaaaaattgaaccattaggagtagcacccaccaaggcaaagagaagagtggtgcagagagaaaaaagagcag
tgggaataggagctttgttccttgggttcttgggagcagcaggaagcactatgggcgcagcctcaatgacgctgacggtacaggccagacaatt
attgtctggtatagtgcagcagcagaacaatttgctgagggctattgaggcgcaacagcatctgttgcaactcacagtctggggcatcaagcagc
tccaggcaagaatcctggctgtggaaagatacctaaaggatcaacagctcctggggatttggggttgctctggaaaactcatttgcaccactgct
gtgccttggaatgctagttggagtaataaatctctggaacagatttggaatcacacgacctggatggagtgggacagagaaattaacaattacac
aagcttaatacactccttaattgaagaatcgcaaaaccagcaagaaaagaatgaacaagaattattggaattagataaatgggcaagtttgtggaa
ttggtttaacataacaaattggctgtggtatataaaattattcataatgatagtaggaggcttggtaggtttaagaatagtttttgctgtactttctatagt
gaatagagttaggcagggatattcaccattatcgtttcagacccacctcccaaccccgaggggacccgacaggcccgaaggaatagaagaag
aaggtggagagagagacagagacagatccattcgattagtgaacggatctcgacggtatcggttaacttttaaaagaaaaggggggattgggg
ggtacagtgcaggggaaagaatagtagacataatagcaacagacatacaaactaaagaattacaaaaacaaattacaaaaattcaaaattttatc
gatcacgagactagcctcgagaagcttgatatcgaattcccacggggttggggttgcgccttttccaaggcagccctgggtttgcgcagggacg
cggctgctctgggcgtggttccgggaaacgcagcggcgccgaccctgggtctcgcacattcttcacgtccgttcgcagcgtcacccggatcttc
gccgctacccttgtgggccccccggcgacgcttcctgctccgcccctaagtcgggaaggttccttgcggttcgcggcgtgccggacgtgacaa
acggaagccgcacgtctcactagtaccctcgcagacggacagcgccagggagcaatggcagcgcgccgaccgcgatgggctgtggccaat
agcggctgctcagcggggcgcgccgagagcagcggccgggaaggggcggtgcgggaggcggggtgtggggcggtagtgtgggccctgt
tcctgcccgcgcggtgttccgcattctgcaagcctccggagcgcacgtcggcagtcggctccctcgttgaccgaatcaccgacctctctcccca
gggggatccaccggtcgccaccatggtgagcaagggcgaggagctgttcaccggggtggtgcccatcctggtcgagctggacggcgacgta
aacggccacaagttcagcgtgtccggcgagggcgagggcgatgccacctacggcaagctgaccctgaagttcatctgcaccaccggcaagc
tgcccgtgccctggcccaccctcgtgaccaccctgacctacggcgtgcagtgcttcagccgctaccccgaccacatgaagcagcacgacttctt
caagtccgccatgcccgaaggctacgtccaggagcgcaccatcttcttcaaggacgacggcaactacaagacccgcgccgaggtgaagttcg
agggcgacaccctggtgaaccgcatcgagctgaagggcatcgacttcaaggaggacggcaacatcctggggcacaagctggagtacaacta
caacagccacaacgtctatatcatggccgacaagcagaagaacggcatcaaggtgaacttcaagatccgccacaacatcgaggacggcagc
gtgcagctcgccgaccactaccagcagaacacccccatcggcgacggccccgtgctgctgcccgacaaccactacctgagcacccagtccg
ccctgagcaaagaccccaacgagaagcgcgatcacatggtcctgctggagttcgtgaccgccgccgggatcactctcggcatggacgagctg
tacaagtaaagcggccgcgtcgacaatcaacctctggattacaaaatttgtgaaagattgactggtattcttaactatgttgctccttttacgctatgt
ggatacgctgctttaatgcctttgtatcatgctattgcttcccgtatggctttcattttctcctccttgtataaatcctggttgctgtctctttatgaggagtt
gtggcccgttgtcaggcaacgtggcgtggtgtgcactgtgtttgctgacgcaacccccactggttggggcattgccaccacctgtcagctcctttc
cgggactttcgctttccccctccctattgccacggcggaactcatcgccgcctgccttgcccgctgctggacaggggctcggctgttgggcactg
acaattccgtggtgttgtcggggaaatcatcgtcctttccttggctgctcgcctgtgttgccacctggattctgcgcgggacgtccttctgctacgtc
ccttcggccctcaatccagcggaccttccttcccgcggcctgctgccggctctgcggcctcttccgcgtcttcgccttcgccctcagacgagtcg
gatctccctttgggccgcctccccgcctggaattcgagctcggtacctttaagaccaatgacttacaaggcagctgtagatcttagccactttttaaa
agaaaaggggggactggaagggctaattcactcccaacgaagacaagatctgctttttgcttgtactgggtctctctggttagaccagatctgagc
ctgggagctctctggctaactagggaacccactgcttaagcctcaataaagcttgccttgagtgcttcaagtagtgtgtgcccgtctgttgtgtgact
ctggtaactagagatccctcagacccttttagtcagtgtggaaaatctctagcagtagtagttcatgtcatcttattattcagtatttataacttgcaaag
aaatgaatatcagagagtgagaggaacttgtttattgcagcttataatggttacaaataaagcaatagcatcacaaatttcacaaataaagcattttttt
cactgcattctagttgtggtttgtccaaactcatcaatgtatcttatcatgtctggctctagctatcccgcccctaactccgcccagttccgcccattct
ccgccccatggctgactaattttttttatttatgcagaggccgaggccgcctcggcctctgagctattccagaagtagtgaggaggcttttttggag
gcctaggcttttgcgtcgagacgtacccaattcgccctatagtgagtcgtattacgcgcgctcactggccgtcgttttacaacgtcgtgactggga
aaaccctggcgttacccaacttaatcgccttgcagcacatccccctttcgccagctggcgtaatagcgaagaggcccgcaccgatcgcccttcc
caacagttgcgcagcctgaatggcgaatggcgcgacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagctctaaatcgg
gggctccctttagggttccgatttagtgctttacggcacctcgaccccaaaaaacttgattagggtgatggttcacgtagtgggccatcgccctgat
agacggtttttcgccctttgacgttggagtccacgttctttaatagtggactcttgttccaaactggaacaacactcaaccctatctcggtctattctttt
gatttataagggattttgccgatttcggcctattggttaaaaaatgagctgatttaacaaaaatttaacgcgaattttaacaaaatattaacgtttacaat
ttcc

### Example 3: Design of Novel Promoter/Enhancer Elements Based on the Human Translocator Protein (TSPO) Putative Regulatory Region

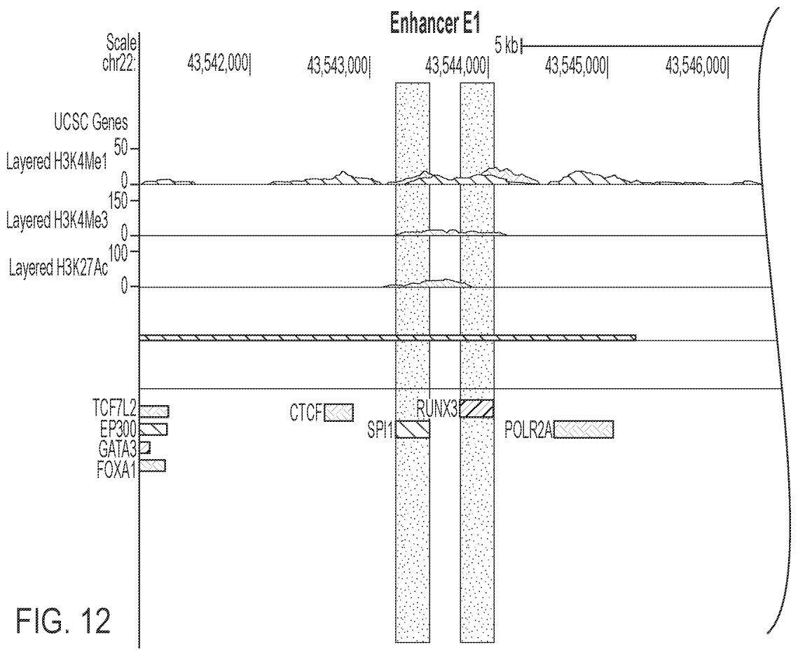

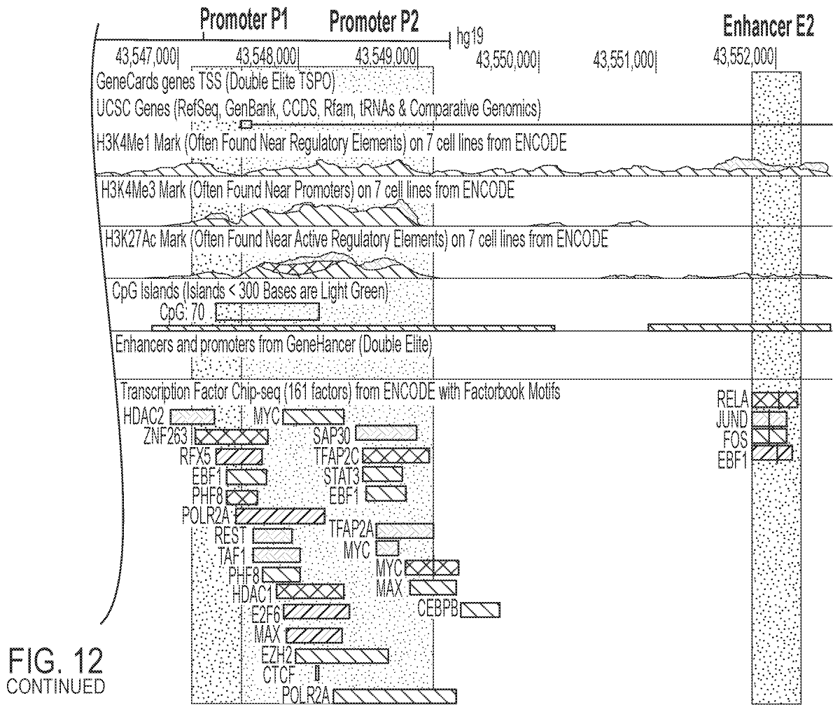

In the genomic region around the human translocator protein (TSPO) Transcriptional Start Site (TSS) on chromosome 22, four clusters of putative transcriptional regulatory elements of the human TSPO gene were identified (i.e., P1, P2, E1, and E2) by integrating publicly-available transcriptional and epigenetic data produced by the Broad Institute-Encode Project using human hematopoietic K652 cells. In particular, regions were identified that were epigenetically marked as active promoters (H3K4me3⁺ and H3K27ac⁺) or active enhancers (H3K4me1⁺ and H3K27ac⁺) bound by hematopoietic-specific transcription factors as detected by Chip-seq genomic maps (FIG. 12). Epigenetically-identified promoter regions were defined as actual promoters if active transcription was confirmed by RNA-seq data.

In summary, two enhancer elements (E1 and E2) and two promotorial regions (P1 and P2) were identified. P2 and E2 were located downstream to the translocator protein (TSPO) transcriptional start site (TSS), within the TSPO gene body, therefore representing a transcribed portion of the translocator protein (TSPO) core promoter (P2) and a putative intragenic enhancer (E2), respectively.

### Example 4: Production of Lentiviral Vectors Carrying Translocator Protein-Based (TSPO-Based) Promoter/Enhancer Elements to Drive Green Fluorescent Protein (GFP) Expression

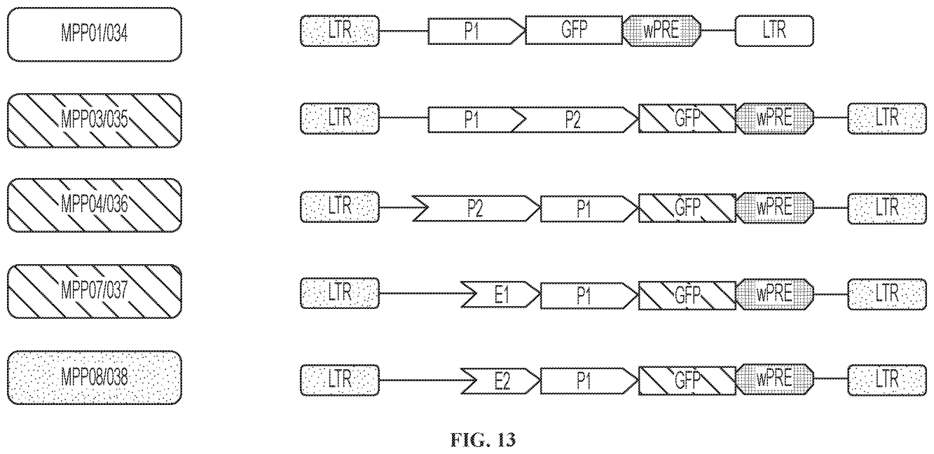

In order to make these putative regulatory elements suitable for cloning into a lentiviral vector system, genomic sequences were modified extensively to eliminate all the canonical and cryptic signals of poly-adenylation, which could lead to premature stop of the lentiviral genome production in the packaging cells, and signals of splicing that could induce transcriptional alterations in the transduced cells by trans-splicing events occurring between the integrated provirus and the host genome. After sequence editing, the four putative regulatory regions (P1, P2, El, and E1) derived from the human translocator protein (TSPO) genomic locus were combined into five regulatory cassettes that were then cloned into five corresponding lentiviral vectors (LVs) to drive the expression of a green fluorescent protein (GFP) reporter gene, see FIG. 13.

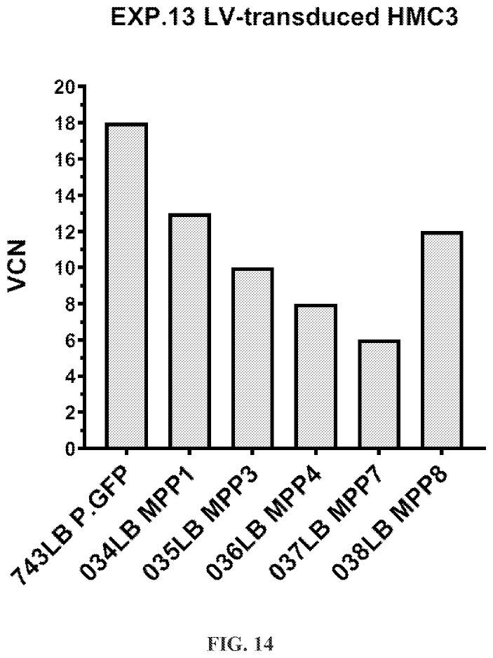

The five lentiviral vectors (LVs) were produced in third-generation lentiviral vectors and pseudotyped with the Vesicular Stomatitis Virus (VSV) g-protein. The resulting viral infectious titers ranged from 5×10⁸ to 1×10⁹ Transducing Units (TU)/mL. Although the different lentiviral vectors (LVs) showed a variable infectivity in human microglial cell lines HMC3, they were all able to efficiently transduce relevant target cells (FIG. 14).

### Example 5: Evaluation of Gene Expression Driven by the Translocator Protein-Based (TSPO-Based) Promoter/Enhancer Elements In Vitro

To test the capability of the five regulatory cassettes to drive the green fluorescent protein (GFP) expression in a similar fashion to the endogenous translocator protein (TSPO) promoter, human microglial HMC3 cells were transduced with the five lentiviral vectors (LVs) and green fluorescent protein (GFP) mRNA and protein expression were analyzed before and after microglial cell-activation.

In vivo, microglia activation is triggered by a plethora of well described subsets of immune receptors such as Toll-like receptors (TLRs), scavenger receptors, and by several cytokine and chemokine receptors. In vitro, HMC3 microglial cells can be activated by incubation with Interferon (IFN)-γ.

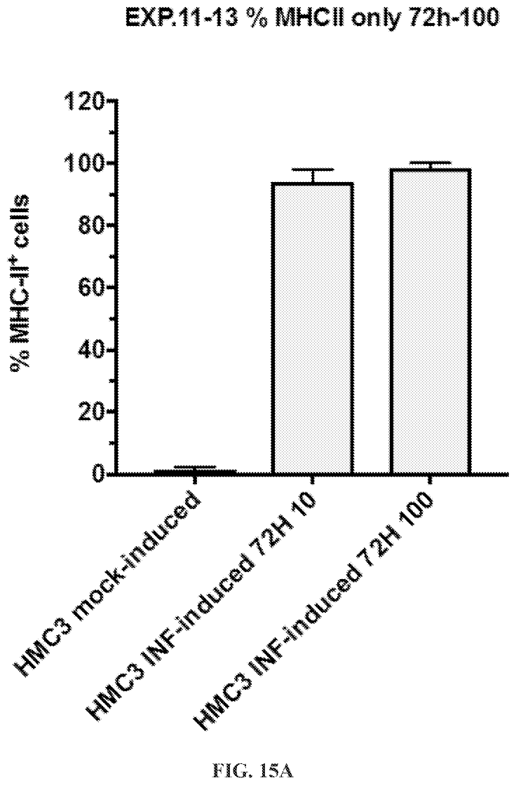

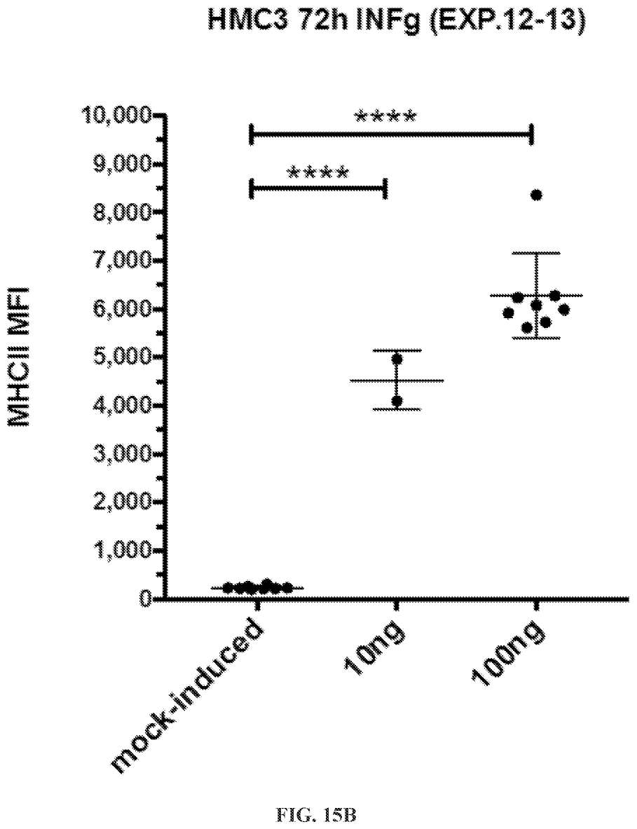

The best results were obtained incubating the cells for 72 hours with 100 ng/ml of IFN-γ (FIGS. 15A and 15B). In response to cell-activation, HMC3 expressed high-levels of Major Histocompatibility Complex (MHC) class-II molecules that were used as positive marker of cell-activation by cytofluorimetric analysis. Activated HMC3 cells expressed the major histocompatibility complex II (MHC-II) surface antigen (FIGS. 15A and 15B).

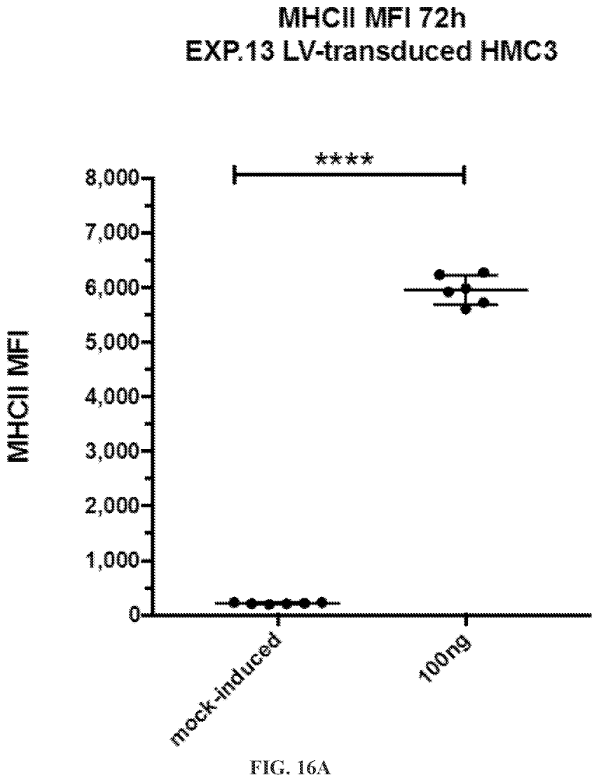

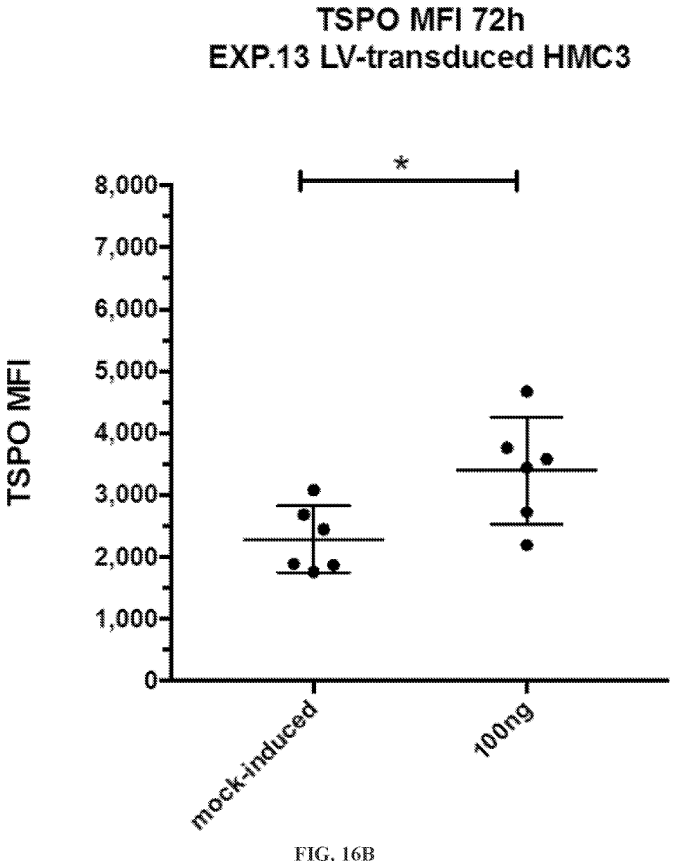

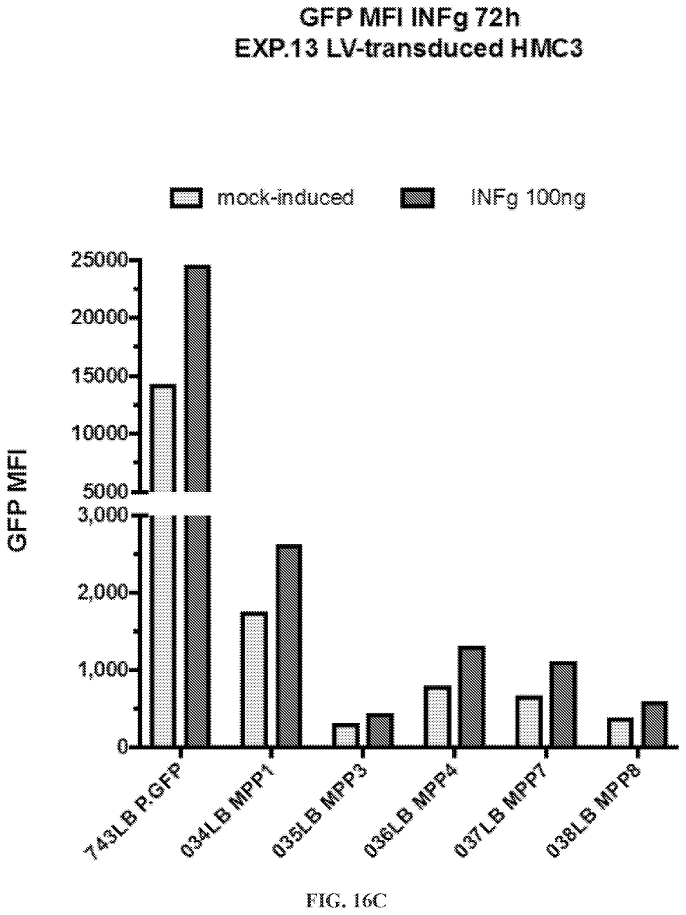

In the HMC3 cells transduced with the different lentiviral vectors (LVs), a concomitant and significant increase of major histocompatibility complex II (MHC-II), translocator protein (TSPO), and green fluorescent protein (GFP) expression was observed upon cell-activation, see FIGS. 16A to 16C.

To precisely evaluate transcriptional regulation driven by the five translocator protein-based (TSPO-based) regulatory cassettes relative to endogenous translocator protein (TSPO) promoter activity, the mRNA level of the lentiviral green fluorescent protein (GFP) and the endogenous translocator protein (TSPO) was quantified and correlated before and after microglial cell-activation. If the lentiviral cassettes were able to closely reproduce the transcriptional regulation operated by the endogenous translocator protein (TSPO) promoter, a similar increase of mRNA expression was observed for the green fluorescent protein (GFP) and the translocator protein (TSPO) transcripts.

HMC3 cells mock-transduced or lentiviral vector-transduced (LV-transduced) cells were activated or not with the IFN-γ. Then, total mRNA was collected, reverse-transcribed and analyzed by real-time polymerase chain reaction (real-time PCR) (with digital-droplet polymerase chain reaction (PCR) technology). Specific primers were designed to quantify the mRNA level of human translocator protein (TSPO), lentiviral green fluorescent protein (GFP) (on the viral Woodchuck hepatitis virus post-transcriptional regulatory element (WPRE) region), and major histocompatibility complex II (MHC-II) (as an additional control) in the same reaction. The resulting total mRNA amounts of the specific genes was normalized to the GAPDH (glyceraldenyde 3-phosphate dehydrogenase) gene and for the specific vector copy number (VCN) in the transduced cells in order to infer the level of transgene expression driven by a single copy of each lentiviral vector (LV) (green fluorescent protein (GFP)/copy).

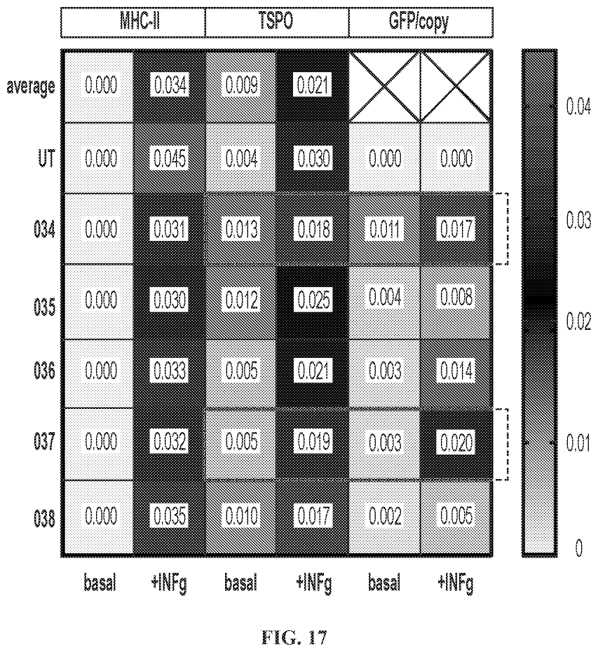

As shown in FIG. 17, the regulatory cassettes contained in the MPP01 (034) and MPPO7 (037) lentiviral vectors (LVs) induced a transgene expression per vector copy comparable to the endogenous translocator protein (TSPO) promoter before and after cell-activation, reproducing quite closely the transcriptional regulation of the endogenous translocator protein (TSPO) promoter.

### Summary of Examples 3 to 5

In vitro data produced in human microglial cell lines for the transcriptional activity of novel translocator protein (TSPO)-based regulatory cassettes, both at the protein and mRNA level, supported efficacy of at least the MPPO1 (034) and MMPO7 (037) lentiviral vector (LV) regulatory cassettes in human relevant cells in vivo to safely and specifically induce a therapeutic level of transgene expression in pathological conditions of the central nervous system (CNS), as in the presence of neuroinflammation.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. This application may be related to PCT/US2020/045106 and PCT/US2017/05677, as well as the PCT applications, entitled “COMPOSITIONS AND METHODS FOR TREATING AMYOTROPHIC LATERAL SCLEROSIS” and “COMPOSITIONS AND METHODS FOR TREATING ALZHEIMER′S DISEASE”, each filed Oct. 1, 2020, which claim priority to the following provisional applications, respectively, 62/908,942 and 62/908,913, all of which are incorporated herein by reference.

1. An isolated translocator protein (TSPO) promoter fragment sufficient to direct expression of a downstream polynucleotide in a microglial cell.

2. The isolated TSPO promoter fragment of claim 1, wherein the TSPO promoter fragment is responsive to inflammation.

3. The isolated TSPO promoter fragment of claim 1, wherein the fragment is selected from the group consisting of about 300, 1200, 1600 nucleotides of a TSPO promoter.

4. The isolated TSPO promoter fragment of claim 1, wherein the fragment comprises or consists of at least about 300 nucleotides.

5. The isolated TSPO promoter fragment of claim 1, wherein the promoter fragment has at least about 85% nucleotide sequence identity to the sequence of MPP01, MPP02, MPP03, or MPP04.

6. The isolated TSPO promoter fragment of claim 1, wherein the full-length promoter comprises nucleotides 83,518,714 to 83,521,414 of murine chromosome 15 or a corresponding portion of a human chromosome.

7-8. (canceled)

9. The isolated TSPO promoter fragment of claim 1, wherein the promoter fragment comprises or consists essentially of a polynucleotide selected from the group consisting of:
>MPP01 (hTSPO_prximal5′prom)(“Pl”)
Tgcatcaccgcgttgcggcctcatcagtcccacgactttgtgcccattttactcatgaggagatggaggcccagagagccagtc
agaaagtggctgggccaggactaagagtgcagcgcgctgcctccgtgccctgcgtcaacagctcaaggaactggggtgctcc
ggaaatggggccaaggctgctgggcagcaggacgctcagggccttggcctcaggagagcaaattccccactcggagatcgg
tcttgttgctgcattttattcatgggaaatctgaggctagaagagacgacaaacgacacgccgttggacacacggcaacgttttag
atgttgggtctggccgggcggccgtcaccggtcaccatggggaggaggaggagccgagagacttgctcgcggccgggggg
aggcagaagcgcgtcccgcgggagaggtggctttgaggagtgagctcccggtcccgcggggacgcgagtgggcccagtgc
ccgggctgccaggcggggcggggcggggccgggcgactgagaggggcggggcctggcggctgggaggggCGGGG
CGGATGCGGGGACAGCGGCCTGGCTAACTCCTGCACGGCAGTGCCCTTCCCG
GAGCGTGCCCTCGCCG
>MPP02(hTSPO_intronic5′prom)(“P2”)
CTGcacagtgaggacgggacgcggagggggcagcgggaacacgccgcccgcatggctgcgacagttggcagcgccgc
gggacagagggaaactgaggccggagccgcagactggacacccgagggggcgacccggggcagcacttggggctcggc
tacgcgcacagggggcggcgggcagcagagtctgggcctccgcggccggggttccaccgccggccgcctccggctcgcg
caacgggagggaaaacttggacaaccctgccacgcccagcccttggccgcgtggcttctcctgctcgaagcgcggtcccagg
agtggccgacgctccctctcctgcccattccgcggatgggcaatcccaggcggaactcccttgagggtctcagaatatctggga
gacctcgggctcttgatctccgagacaccccgtttcgtagtggagaacagtccagatcggggaagtttattttgcccaaagccgc
atagaggccccctggccctcgattccctctgcggggctcagcagcgttgcagcctagacgggtcttactgtgagccgagcagc
ctctgggaccacagaccttcccctaccccaacgttagaagccggagcccagcaaggagaagcgcgcacctcctgctgtgaac
gcgcacgacgccagggcagctgccagaggccatggcctggcgtgggcctggagcccctctggccagcctgcacggggcca
gggctacgggataccagcagcgtgccctgggctggatggcaggagagacaggacttgaggctgtcccagaatgggctcagg
cagggcgaggatatcaggggaggtggtgtacaggaagcagccgcccagcttgcctggcacacagcaagccctgcccatgaa
ggcctactgccagaacagtgggcgaggcccggcgtctctgtggagtcggtggggcccgggacagggcagcctgaggcagg
tttccactggcggtgaaaggggccgtgtggcaaggacaggagagccagcctcagcccagcaggggaaggcggcccctgag
tctccacctggctgctggcagccccactcggagcatcggcgaaactgaggcttgccaaagaagcctttgtccagagtcacgca
gctggcgcggtggagccagggccagaacccgtgcaggctgatcccagcctgccttctccactgtgccccg
>MPP03(hTSPO_upstream_plus_intronic_prom)(“P1 + P2”)
GAGtgcatcaccgcgttgcggcctcatcagtcccacgactttgtgcccattttactcatgaggagatggaggcccagagagcc
agtcagaaagtggctgggccaggactaagagtgcagcgcgctgcctccgtgccctgcgtcaacagctcaaggaactggggtg
ctccggaaatggggccaaggctgctgggcagcaggacgctcagggccttggcctcaggagagcaaattccccactcggagat
cggtcttgttgctgcattttattcatgggaaatctgaggctagaagagacgacaaacgacacgccgttggacacacggcaacgttt
tagatgttgggtctggccgggcggccgtcaccggtcaccatggggaggaggaggagccgagagacttgctcgcggccggg
gggaggcagaagcgcgtcccgcgggagaggtggctttgaggagtgagctcccggtcccgcggggacgcgagtgggccca
gtgcccgggctgccaggcggggcggggcggggccgggcgactgagaggggcggggcctggcggctgggaggggCG
GGGCGGATGCGGGGACAGCGGCCTGGCTAACTCCTGCACGGCAGTGCCCTTC
CCGGAGCGTGCCCTCGCCGCTGcacagtgaggacgggacgcggagggggcagcgggaacacgccgcc
cgcatggctgcgacagttggcagcgccgcgggacagagggaaactgaggccggagccgcagactggacacccgaggggg
cgacccggggcagcacttggggctcggctacgcgcacagggggcggcgggcagcagagtctgggcctccgcggccgggg
ttccaccgccggccgcctccggctcgcgcaacgggagggaaaacttggacaaccctgccacgcccagcccttggccgcgtg
gcttctcctgctcgaagcgcggtcccaggagtggccgacgctccctctcctgcccattccgcggatgggcaatcccaggcgga
actcccttgagggtctcagaatatctgggagacctcgggctcttgatctccgagacaccccgtttcgtagtggagaacagtccag
atcggggaagtttattttgcccaaagccgcatagaggccccctggccctcgattccctctgcggggctcagcagcgttgcagcct
agacgggtcttactgtgagccgagcagcctctgggaccacagaccttcccctaccccaacgttagaagccggagcccagcaa
ggagaagcgcgcacctcctgctgtgaacgcgcacgacgccagggcagctgccagaggccatggcctggcgtgggcctgga
gcccctctggccagcctgcacggggccagggctacgggataccagcagcgtgccctgggctggatggcaggagagacagg
acttgaggctgtcccagaatgggctcaggcagggcgaggatatcaggggaggtggtgtacaggaagcagccgcccagcttg
cctggcacacagcaagccctgcccatgaaggcctactgccagaacagtgggcgaggcccggcgtctctgtggagtcggtgg
ggcccgggacagggcagcctgaggcaggtttccactggcggtgaaaggggccgtgtggcaaggacaggagagccagcctc
agcccagcaggggaaggcggcccctgagtctccacctggctgctggcagccccactcggagcatcggcgaaactgaggctt
gccaaagaagcctttgtccagagtcacgcagctggcgcggtggagccagggccagaacccgtgcaggctgatcccagcctg
ccttctccactgtgccccg
>MPP04(hTSPO_intronic_plus_upstream_prom)(“P2 + Pl”)
CTGcacagtgaggacgggacgcggagggggcagcgggaacacgccgcccgcatggctgcgacagttggcagcgccgc
gggacagagggaaactgaggccggagccgcagactggacacccgagggggcgacccggggcagcacttggggctcggc
tacgcgcacagggggcggcgggcagcagagtctgggcctccgcggccggggttccaccgccggccgcctccggctcgcg
caacgggagggaaaacttggacaaccctgccacgcccagcccttggccgcgtggcttctcctgctcgaagcgcggtcccagg
agtggccgacgctccctctcctgcccattccgcggatgggcaatcccaggcggaactcccttgagggtctcagaatatctggga
gacctcgggctcttgatctccgagacaccccgtttcgtagtggagaacagtccagatcggggaagtttattttgcccaaagccgc
atagaggccccctggccctcgattccctctgcggggctcagcagcgttgcagcctagacgggtcttactgtgagccgagcagc
ctctgggaccacagaccttcccctaccccaacgttagaagccggagcccagcaaggagaagcgcgcacctcctgctgtgaac
gcgcacgacgccagggcagctgccagaggccatggcctggcgtgggcctggagcccctctggccagcctgcacggggcca
gggctacgggataccagcagcgtgccctgggctggatggcaggagagacaggacttgaggctgtcccagaatgggctcagg
cagggcgaggatatcaggggaggtggtgtacaggaagcagccgcccagcttgcctggcacacagcaagccctgcccatgaa
ggcctactgccagaacagtgggcgaggcccggcgtctctgtggagtcggtggggcccgggacagggcagcctgaggcagg
tttccactggcggtgaaaggggccgtgtggcaaggacaggagagccagcctcagcccagcaggggaaggcggcccctgag
tctccacctggctgctggcagccccactcggagcatcggcgaaactgaggcttgccaaagaagcctttgtccagagtcacgca
gctggcgcggtggagccagggccagaacccgtgcaggctgatcccagcctgccttctccactgtgccccgtgcatcaccgcgt
tgcggcctcatcagtcccacgactttgtgcccattttactcatgaggagatggaggcccagagagccagtcagaaagtggctgg
gccaggactaagagtgcagcgcgctgcctccgtgccctgcgtcaacagctcaaggaactggggtgctccggaaatggggcca
aggctgctgggcagcaggacgctcagggccttggcctcaggagagcaaattccccactcggagatcggtcttgttgctgcatttt
attcatgggaaatctgaggctagaagagacgacaaacgacacgccgttggacacacggcaacgttttagatgttgggtctggcc
gggcggccgtcaccggtcaccatggggaggaggaggagccgagagacttgctcgcggccggggggaggcagaagcgcg
tcccgcgggagaggtggctttgaggagtgagctcccggtcccgcggggacgcgagtgggcccagtgcccgggctgccagg
cggggcggggcggggccgggcgactgagaggggcggggcctggcggctgggaggggCGGGGCGGATGCG
GGGACAGCGGCCTGGCTAACTCCTGCACGGCAGTGCCCTTCCCGGAGCGTGC
CCTCGCCGGGATCC

10-11. (canceled)

12. An expression vector comprising the promoter fragment of claim 1.

13-17. (canceled)

18. The expression vector of claim 17, wherein the vector further comprises an enhancer that comprises or consists essentially of E1, E1.1, E1.2, or E2.

19. An expression vector comprising a regulatory sequence driving expression of a downstream gene, the regulatory sequence selected from the group consisting of P1, from 5′ to 3′ P1 and P2, from 5′ to 3′ P2 and P1, from 5′ to 3′ El and P1, and 5′ to 3′ E2 and P1.

20-23. (canceled)

24. An expression vector comprising a regulatory element comprising a polynucleotide sequence having at least 85% identity to any of the following sequences:
>MPP01(hTSPO_prximal5′prom)(“Pl”)
Tgcatcaccgcgttgcggcctcatcagtcccacgactttgtgcccattttactcatgaggagatggaggcccagagagccagtc
agaaagtggctgggccaggactaagagtgcagcgcgctgcctccgtgccctgcgtcaacagctcaaggaactggggtgctcc
ggaaatggggccaaggctgctgggcagcaggacgctcagggccttggcctcaggagagcaaattccccactcggagatcgg
tcttgttgctgcattttattcatgggaaatctgaggctagaagagacgacaaacgacacgccgttggacacacggcaacgttttag
atgttgggtctggccgggcggccgtcaccggtcaccatggggaggaggaggagccgagagacttgctcgcggccgggggg
aggcagaagcgcgtcccgcgggagaggtggctttgaggagtgagctcccggtcccgcggggacgcgagtgggcccagtgc
ccgggctgccaggcggggcggggcggggccgggcgactgagaggggcggggcctggcggctgggaggggCGGGG
CGGATGCGGGGACAGCGGCCTGGCTAACTCCTGCACGGCAGTGCCCTTCCCG
GAGCGTGCCCTCGCCG.
>MPP02(hTSPO_intronic5'prom)(“P2”)
CTGcacagtgaggacgggacgcggagggggcagcgggaacacgccgcccgcatggctgcgacagttggcagcgccgc
gggacagagggaaactgaggccggagccgcagactggacacccgagggggcgacccggggcagcacttggggctcggc
tacgcgcacagggggcggcgggcagcagagtctgggcctccgcggccggggttccaccgccggccgcctccggctcgcg
caacgggagggaaaacttggacaaccctgccacgcccagcccttggccgcgtggcttctcctgctcgaagcgcggtcccagg
agtggccgacgctccctctcctgcccattccgcggatgggcaatcccaggcggaactcccttgagggtctcagaatatctggga
gacctcgggctcttgatctccgagacaccccgtttcgtagtggagaacagtccagatcggggaagtttattttgcccaaagccgc
atagaggccccctggccctcgattccctctgcggggctcagcagcgttgcagcctagacgggtcttactgtgagccgagcagc
ctctgggaccacagaccttcccctaccccaacgttagaagccggagcccagcaaggagaagcgcgcacctcctgctgtgaac
gcgcacgacgccagggcagctgccagaggccatggcctggcgtgggcctggagcccctctggccagcctgcacggggcca
gggctacgggataccagcagcgtgccctgggctggatggcaggagagacaggacttgaggctgtcccagaatgggctcagg
cagggcgaggatatcaggggaggtggtgtacaggaagcagccgcccagcttgcctggcacacagcaagccctgcccatgaa
ggcctactgccagaacagtgggcgaggcccggcgtctctgtggagtcggtggggcccgggacagggcagcctgaggcagg
tttccactggcggtgaaaggggccgtgtggcaaggacaggagagccagcctcagcccagcaggggaaggcggcccctgag
tctccacctggctgctggcagccccactcggagcatcggcgaaactgaggcttgccaaagaagcctttgtccagagtcacgca
gctggcgcggtggagccagggccagaacccgtgcaggctgatcccagcctgccttctccactgtgccccg
>E1
agactctgtctcaaaaaaaaaaaaaaaaaaaaaaaaagagagagagaaaaaggaagttagaaaaacagccctagaggcccta
cattctgagtaataggagttccagaaaggaagtgattgctgcacaacataaatttgaaaagaaagagaagtgagaaaatagagg
gaaggaaatcaaagaaataatccaacttctgaaaagtaaagaatgagcttccagcgggaaagtgcctgttgagtgcaggcaca
gtggaggaaatgaagctgggtgtgttgccaggagttggaaacttgtctgagcaacatagcgcgaccctgtgtctacaaaaaaat
aaaaacaaaacaaaaaacaaccaaagacttccgaaacagaatggctttagcctgctcaaccgcacactggcacctggccaaca
gcatctcttcatgattctgaaggacaacgatctgcagctcagccaagcatcagccatctatggcctaggatgcaagaattcagca
atgttaccttcAgactctgtctcaaaaaaaaaaaaaaaaaaaaaaaaagagagagagaaaaaggaagttagaaaaacagccct
agaggccctacattctgagtaataggagttccagaaaggaagtgattgctgcacaacataaatttgaaaagaaagagaagtgag
aaaatagagggaaggaaatcaaagaaataatccaacttctgaaaagtaaagaatgagcttccagcgggaaagtgcctgttgagt
gcaggcacagtggaggaaatgaagctgggtgtgttgccaggagttggaaacttgtctgagcaacatagcgcgaccctgtgtcta
caaaaaaataaaaacaaaacaaaaaacaaccaaagacttccgaaacagaatggctttagcctgctcaaccgcacactggcacc
tggccaacagcatctcttcatgattctgaaggacaacgatctgcagctcagccaagcatcagccatctatggcctaggatgcaag
aattcagcaatgttaccttctgcatcaccgcgttgcggcctcatcagtcccacgactttgtgcccattttactcatgaggagatggag
gcccagagagccagtcagaaagtggctgggccaggactaagagtgcagcgcgctgcctccgtgccctgcgtcaacagctca
aggaactggggtgctccggaaatggggccaaggctgctgggcagcaggacgctcagggccttggcctcaggagagcaaatt
ccccactcggagatcggtcttgttgctgcattttattcatgggaaatctgaggctagaagagacgacaaacgacacgccgttgga
cacacggcaacgttttagatgttgggtctggccgggcggccgtcaccggtcaccatggggaggaggaggagccgagagactt
gctcgcggccggggggaggcagaagcgcgtcccgcgggagaggtggctttgaggagtgagctcccggtcccgcggggac
gcgagtgggcccagtgcccgggctgccaggcggggcggggcggggccgggcgactgagaggggcggggcctggcggc
tgggaggggCGGGGCGGATGCGGGGACAGCGGCCTGGCTAACTCCTGCACGGCA
GTGCCCTTCCCGGAGCGTGCCCTCGCCG
>E2
Tgcatcaccgcgttgcggcctcatcagtcccacgactttgtgcccattttactcatgaggagatggaggcccagagagccagtc
agaaagtggctgggccaggactaagagtgcagcgcgctgcctccgtgccctgcgtcaacagctcaaggaactggggtgctcc
ggaaatggggccaaggctgctgggcagcaggacgctcagggccttggcctcaggagagcaaattccccactcggagatcgg
tcttgttgctgcattttattcatgggaaatctgaggctagaagagacgacaaacgacacgccgttggacacacggcaacgttttag
atgttgggtctggccgggcggccgtcaccggtcaccatggggaggaggaggagccgagagacttgctcgcggccgggggg
aggcagaagcgcgtcccgcgggagaggtggctttgaggagtgagctcccggtcccgcggggacgcgagtgggcccagtgc
ccgggctgccaggcggggcggggcggggccgggcgactgagaggggcggggcctggcggctgggaggggCGGGG
CGGATGCGGGGACAGCGGCCTGGCTAACTCCTGCACGGCAGTGCCCTTCCCG
GAGCGTGCCCTCGCCG.
>MPP05A(hTSPO upstream enhancer)(“E1.1”)
agactctgtctcaaaaaaaaaaaaaaaaaaaaaaaaagagagagagaaaaaggaagttagaaaaacagccctagaggcccta
cattctgagtaataggagttccagaaaggaagtgattgctgcacaacataaatttgaaaagaaagagaagtgagaaaatagagg
gaaggaaatcaaagaaataatccaacttctgaaaagtaaagaatgagcttccagcgggaaagtgcctgttgagtgcaggcaca
gtggaggaaatgaagctgggtgtgttgccaggagttggaaacttgtctgagcaacatagcgcgaccctgtgtctacaaaaaaat
aaaaacaaaacaaaaaacaaccaaagacttccgaaacagaatggctttagcctgctcaaccgcacactggcacctggccaaca
gcatctcttcatgattctgaaggacaacgatctgcagctcagccaagcatcagccatctatggcctaggatgcaagaattcagca
atgttaccttc
>MPP05B(hTSPO_upstream_enhancer)(“E1.2”)
Agactctgtctcaaaaaaaaaaaaaaaaaaaaaaaaagagagagagaaaaaggaagttagaaaaacagccctagaggccct
acattctgagtaataggagttccagaaaggaagtgattgctgcacaacataaatttgaaaagaaagagaagtgagaaaatagag
ggaaggaaatcaaagaaataatccaacttctgaaaagtaaagaatgagcttccagcgggaaagtgcctgttgagtgcaggcac
agtggaggaaatgaagctgggtgtgttgccaggagttggaaacttgtctgagcaacatagcgcgaccctgtgtctacaaaaaaa
taaaaacaaaacaaaaaacaaccaaagacttccgaaacagaatggctttagcctgctcaaccgcacactggcacctggccaac
agcatctcttcatgattctgaaggacaacgatctgcagctcagccaagcatcagccatctatggcctaggatgcaagaattcagc
aatgttaccttctgcatcaccgcgttgcggcctcatcagtcccacgactttgtgcccattttactcatgaggagatggaggcccaga
gagccagtcagaaagtggctgggccaggactaagagtgcagcgcgctgcctccgtgccctgcgtcaacagctcaaggaactg
gggtgctccggaaatggggccaaggctgctgggcagcaggacgctcagggccttggcctcaggagagcaaattccccactc
ggagatcggtcttgttgctgcattttattcatgggaaatctgaggctagaagagacgacaaacgacacgccgttggacacacggc
aacgttttagatgttgggtctggccgggcggccgtcaccggtcaccatggggaggaggaggagccgagagacttgctcgcgg
ccggggggaggcagaagcgcgtcccgcgggagaggtggctttgaggagtgagctcccggtcccgcggggacgcgagtgg
gcccagtgcccgggctgccaggcggggcggggcggggccgggcgactgagaggggcggggcctggcggctgggaggg
gCGGGGCGGATGCGGGGACAGCGGCCTGGCTAACTCCTGCACGGCAGTGCCC
TTCCCGGAGCGTGCCCTCGCCG

25. A mammalian cell comprising the expression vector of claim 12.

26-27. (canceled)

28. A method for expressing a therapeutic polypeptide in a cell, the method comprising contacting the cell with an expression vector comprising the promoter fragment of claim 1.

29. (canceled)

30. A method of treating a subject having a neurological disease, the method comprising contacting a cell of the subject with an expression vector comprising the promoter fragment of claim 1 or administering to the subject a cell comprising an expression vector having the promoter fragment of claim 1.

31. (canceled)

32. The method of claim 30, wherein the neurological disease is Alzheimer's disease, amyotrophic lateral sclerosis, metachromatic leuokodystrophy, adrenoleukodystophy, a lysosomal storage disorder, or Parkinson's disease.

33-36. (canceled)

37. The method of claim 30, wherein the promoter fragment comprises or consists essentially of P1 or P1+P2.

38. (canceled)

39. The method of claim 30, wherein the vector further comprises an enhancer comprising or consisting essentially of E1, E2m .

40-41. (canceled)

42. The method of claim 30, wherein the cell is a hematopoietic stem cell or progenitor thereof or a microglial cell.

43. The method of claim 30, wherein the subject undergoes ablative conditioning prior to the method.

44. (canceled)

48. The method of claim 30, wherein the cell is a cell in vitro or in vivo.

49. A pharmaceutical composition comprising the expression vector of claim 12 or a cell comprising said vector.

50. (canceled)
